(19) United States
(12) Patent Application Publication
(10) Pub. No.: US 2023/0330110 A1
(43) Pub. Date: Oct. 19, 2023

(54) HARD CAPSULE DOSAGE FORM AND USES THEREOF

(71) Applicant: EIRGEN PHARMA LTD., Waterford (IE)

(72) Inventors: Reem Elamein Elsiddig, Waterford (IE); Colm Nulty, Waterford (IE)

(21) Appl. No.: 18/199,117

(22) Filed: May 18, 2023

Related U.S. Application Data
(63) Continuation of application No. 18/005,946, filed on Jan. 18, 2023.
Application No. 18/005,946 is a continuation of application No. PCT/IB21/00220, filed on Apr. 6, 2021.
(60) Provisional application No. 63/032,714, filed on May 31, 2020.

(51) Int. Cl. A61K 9/48; A61K 31/593

(52) U.S. Cl. CPC A61K 9/4816; A61K 31/593; A61K 9/4858; A61K 9/4866; A61K 9/485

(57) ABSTRACT

Provided herein are hard capsule dosage forms containing a vitamin D compound, and methods of making and using the same.

### CROSS-REFERENCE TO RELATED APPLICATIONS

This is a continuation of U.S. patent application Ser. No. 18/005,946, filed Jan. 18, 2023, which is a 371 national phase of PCT/IB21/00376 filed May 31, 2021, which is a continuation of PCT application PCT/IB21/00220 filed Apr. 6, 2021, and claims the benefit of under 35 U.S.C. § 119(e) of U.S. Provisional Patent Application No. 63/032,714, filed May 31, 2020. The disclosures of the foregoing applications are hereby incorporated by reference herein.

### BACKGROUND

### Field of the Disclosure

The disclosure relates generally to a dosage form suitable for use with vitamin D active compounds, e.g. to a hard capsule formulation for delivering 25-hydroxyvitamin D compounds, and uses thereof in treatment.

### Brief Description of Related Technology

Extended release calcifediol (ERC) dosage forms and related methods have been described, e.g. in U.S. Pat. Nos. 8,2207,149, 8,426,391, 9,861,644, and U.S. Patent Application Publication No. 2019/0083513 A1, and international patent application publication WO 2020/044314 A1, the entire disclosures of which are incorporated herein by reference. An FDA-approved product is marketed as Rayaldee® (calcifediol) extended-release capsules in soft vegetable-based (Optishell®) capsules.

### SUMMARY

One aspect of the disclosure is a hard capsule dosage form comprising a hard shell capsule containing a solid or semi-solid composition comprising a 25-hydroxyvitamin D compound, the hard shell capsule comprising a cellulose ether and a gelatinizing agent.

Another aspect of the disclosure is a hard capsule dosage form comprising a hard shell capsule containing a solid or semi-solid composition comprising a 25-hydroxyvitamin D compound, the hard shell capsule comprising a cellulose ether.

Another aspect of the disclosure is a method of delivering 25-hydroxyvitamin D or calcifediol to a subject in need thereof, comprising administering a hard shell capsule dosage form of the disclosure herein to the subject. Similarly, an aspect of the disclosure is use of a hard shell capsule dosage form according to the disclosure herein for administration to a subject in need thereof. The subject in need can be any one in need of 25-hydroxyvitamin D, or one having a disease or condition described herein.

Another aspect of the disclosure is use of a gelatinized hard capsule dosage form of the disclosure herein, wherein the dosage form releases no more than about 5% of the 25-hydroxyvitamin D or calcifediol in the formulation contained in the dosage form in two hours in an acidic medium, optionally pH 1.2, or pH 1.5, and further optionally at 37° C., to contain a composition comprising 25-hydroxyvitamin D or calcifediol and provide increased recovery and/or reduced degradation of said 25-hydroxyvitamin D or calcifediol after exposure of the dosage form to acidic conditions.

Another aspect of the disclosure is use of a gelatinized hard capsule dosage form of the disclosure herein, wherein the dosage form releases no more than about 5% of the 25-hydroxyvitamin D or calcifediol in the formulation contained in the dosage form in two hours in an acidic medium, optionally pH 1.2, or pH 1.5, and further optionally at 37° C., to contain a composition comprising 25-hydroxyvitamin D or calcifediol and for oral administration to a mammal.

4 Another aspect of the disclosure is use of a gelatinized hard capsule dosage form of the disclosure herein, wherein the dosage form releases no more than about 5% of the 25-hydroxyvitamin D or calcifediol in the formulation contained in the dosage form in two hours in an acidic medium, optionally pH 1.2, or pH 1.5, and further optionally at 37° C., to contain a composition comprising 25-hydroxyvitamin D or calcifediol and expose the dosage form to acidic conditions, optionally less than pH 4.5, and optionally less than pH 3.5.

Another aspect of the disclosure is a method of making a hard shell capsule dosage form according to the disclosure herein, including disposing a formulation containing 25-hydroxyvitamin D in a gelatinized hypromellose hard shell capsule according to the disclosure herein. The 25-hydroxyvitamin D formulation optionally can be heated before disposing it in the shell, e.g. if it is a solid or semi-solid formulation which flows more readily when heated. The shell optionally can have a sealing solution applied thereto, e.g. a hypromellose sealing solution.

For the compositions and methods described herein, optional features, including but not limited to components, compositional ranges thereof, substituents, conditions, and steps, are contemplated to be selected from the various aspects, embodiments, and examples provided herein.

Further aspects and advantages will be apparent to those of ordinary skill in the art from a review of the following detailed description, taken in conjunction with the drawings. While the methods, uses, and compositions are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

To further facilitate the understanding of the present invention, eight figures are attached hereto.

FIG. 1 shows mean serum concentration of 25-hydroxyvitamin D₃ curves after oral administration of 900 mcg of modified release calcifediol soft capsules.

FIG. 2 shows in vitro dissolution profiles of hard capsule dosage forms according to the disclosure.

FIG. 3 shows serum total 25-D concentrations as a function of time with repeated dosing of ERC (Rayaldee® (calcifediol) extended-release capsules), IR calcifediol, high-dose cholecalciferol, and paricalcitol plus low-dose cholecalciferol in adult patients with secondary hyperparathyroidism (SHPT), stage 3 or 4 chronic kidney disease (CKD) and vitamin D insufficiency according to Example 3.

FIG. 4 shows VMR as a function of time with repeated dosing of ERC (Rayaldee® (calcifediol) extended-release capsules), IR calcifediol, high-dose cholecalciferol, and paricalcitol plus low-dose cholecalciferol in adult patients with secondary hyperparathyroidism (SHPT), stage 3 or 4 chronic kidney disease (CKD) and vitamin D insufficiency according to Example 3.

FIGS. 5A and 5B show serum total 25-hydroxyvitamin D response rates with repeated dosing of ERC (Rayaldee® (calcifediol) extended-release capsules), IR calcifediol, high-dose cholecalciferol, and paricalcitol plus low-dose cholecalciferol in adult patients with secondary hyperparathyroidism (SHPT), stage 3 or 4 chronic kidney disease (CKD) and vitamin D insufficiency according to Example 3.

FIGS. 6A, 6B and 6A show Plasma iPTH-lowering responses with repeated dosing of ERC (Rayaldee® (calcifediol) extended-release capsules), IR calcifediol, high-dose cholecalciferol, and paricalcitol plus low-dose cholecalciferol in adult patients with secondary hyperparathyroidism (SHPT), stage 3 or 4 chronic kidney disease (CKD) and vitamin D insufficiency according to Example 3.

FIG. 7 shows dissolution profiles for hypromellose capsule samples in pH 6.8 medium (left) and in a two stage dissolution procedure (right) according to Example 5.

FIG. 8 shows dissolution profiles for vegetable capsule samples in pH 6.8 medium (left), a two stage dissolution procedure (middle), and pH 1.2 medium (right/bottom).

FIGS. 9 and 10 show dissolution profiles as described in Example 6, in pH 1.2 and pH 4.5 buffers, respectively.

FIG. 11 shows two stage dissolution profiles as described in Example 7.

### DESCRIPTION

The dosage form of the present disclosure is hard capsule formulation for a vitamin D compound, e.g. 25-hydroxyvitamin D While the general description below describes compositions and uses with 25-hydroxyvitamin D, it is contemplated that in each instance another vitamin D compound can be used in place of a 25-hydroxyvitamin D compound. It is also contemplated that in each instance when 25-hydroxyvitamin D is mentioned, calcifediol can be the specific 25-hydroxyvitamin D compound. Another aspect of the disclosure herein is an extended-release hard capsule formulation containing a 25-hydroxyvitamin D compound, e.g. for oral administration. The formulation can optionally also have delayed release characteristics. In any of the methods described herein, the 25-hydroxyvitamin D compound(s) can be administered in the form of the hard capsule formulation as described herein.

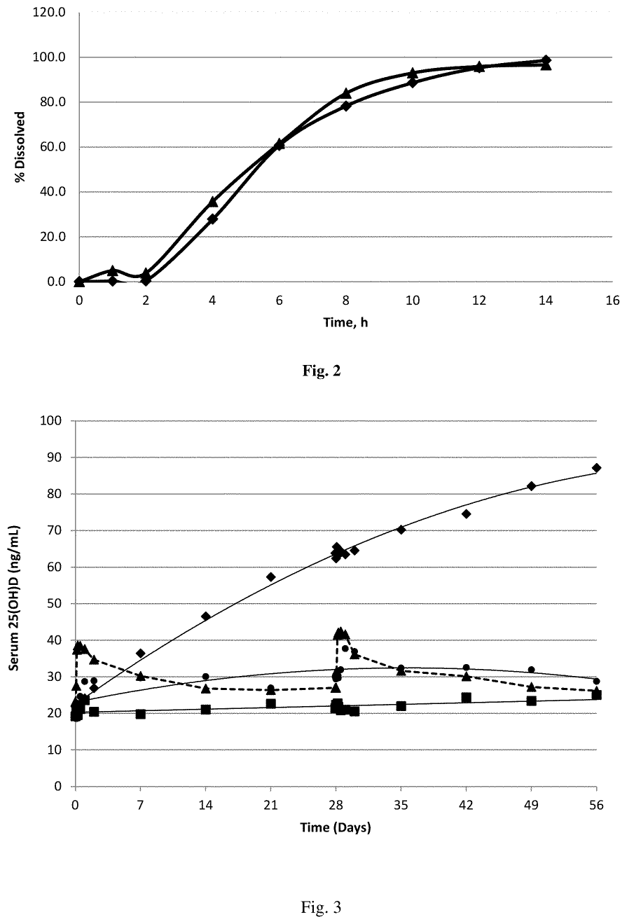

It was surprisingly found that wax-based formulations such as those used in Rayaldee® (calcifediol) extended-release capsules, when placed in hard gelatin capsules and hard HPMC capsules, did not provide a matching dissolution release profile using a two-stage dissolution method (2 hours at pH 1.2, then transfer to pH 6.8 buffered medium), especially in the 0-2 hour and 0-4 hour time ranges. However, using a gelatinized HPMC hard capsule shell, such a dosage form could be made to have a dissolution profile closely matching that of Rayaldee® (calcifediol) extended-release capsules. See FIG. 2. It was also found that the hypromellose-based hard capsule shell, while providing a more consistent rupture time compared to softgel capsules, provided an earlier rupture time. Accordingly, to achieve a dissolution release profile more closely matching Rayaldee® (calcifediol) extended-release capsules, the amount of wax is increased to slow the rate of release of the active.

For the compositions and methods described herein, optional features, including but not limited to components, compositional ranges thereof, substituents, conditions, and steps, are contemplated to be selected from the various aspects, embodiments, and examples provided herein. While the methods and compositions described herein are capable of embodiments in various forms, the description hereafter includes specific embodiments with the understanding that the disclosure is illustrative, and is not intended to limit the invention to the specific embodiments described herein.

U.S. Pat. Nos. 5,264,223 and 5,431,917 describe capsules produced by the use of HPMC with a gelatinizing agent such as carrageenan. The production of such capsules were claimed to occur under similar temperature setting as that of gelatin capsules. Shionogi Qualicaps Co. (Japan) produces a HPMC capsule containing carrageenan as a gelling aid (e.g. kappa- and/or iota-carrageenans) and potassium chloride as gelation promoter. U.S. Pat. No. 6,410,050 B1 describes cellulose capsules (including HPMC) containing pectin and glycerin. U.S. Pat. No. 6,517,865 B2 describes HPMC capsules with hydrocolloids such as gellan gum and sequestering agents (such as EDTA, sodium citrate, and citric acid). For example, it describes a capsule material having 90 to 99.98% by weight of at least one cellulose ether having a water content of 2 to 10% and a viscosity of 3 to 15 cps measured in a 2% aqueous solution at 20° C.; 0.01 to 5% by weight of gellan gum; and 0.01 to 8% by weight of a sequestering agent selected from the group consisting of EDTA, sodium citrate, citric acid and combinations thereof. For example, it is contemplated to use a HPMC capsule containing at least one cellulose ether, optionally HPMC, having a water content of 2 to 10% and a viscosity of 3 to 15 cps measured in a 2% aqueous solution at 20° C. and a gelling agent, optionally gellan gum, in an amount of about 0.01 to about 10% by weight, or about 1% to about 8%, or about 2% to about 7%, or about 4% to about 6%, or about 5% by weight. Such gelatinized HPMC capsules are believed to provide a slower rupture or disintegration time, e.g. in the stomach, compared to HPMC capsules without a gelatinizing aid. In addition or in the alternative, the HPMC capsules can comprise an enteric coating to retard or prevent dissolution or disintegration of the capsule shell in the gastric environment. Enteric coating materials which resist dissolution in acidic media and dissolve in neutral and alkaline media are known and include, for example, methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, methyl methacrylate-methacrylic acid copolymers, shellac, cellulose acetate trimellitate, sodium alginate, zein, and a coating solution including a mixture of ethylcellulose, medium chain triglycerides, oleic acid, sodium alginate, and stearic acid. A hard capsule without a gelatinizing agent or with small percentages (e.g. 0-4% w/w) thereof may optionally be enteric coated to achieve minimal to zero release in the 0-2 hour time period after dosing.

The hard shell capsule is primarily based on a cellulose ether or mixture thereof. The hard shell capsule of the disclosure herein is not a gelatin-based capsule. Suitable cellulose ethers are alkyl and/or hydroxyalkyl substituted cellulose ether with 1 to 4 carbon atoms in the alkyl chains, e.g. methyl cellulose, hydroxyethyl cellulose, hydroxypropyl cellulose, hydroxyethylmethyl cellulose, hydroxyethylethyl cellulose, hydroxypropylmethyl cellulose or the like. Hydroxypropylmethyl cellulose is commonly known as hypromellose, and is particularly contemplated. Various examples and embodiments are described herein in connection with hypromellose, and in each instance it is more generally contemplated that the hard shell capsule can be based on another cellulose ether or, mixture thereof, as described herein. The amount of the cellulose ether or mixture of cellulose ethers can be, for example, greater than 50% by weight of the hard shell capsule, or at least 60%, or at least 70%, or at least 80%, or at least 90%, for example 95% to 99.98% by weight of the hard shell capsule. The viscosity of the cellulose ether or blend can be in a range of 3 cps to 15 cps in 2% aqueous solution at 20° C., or in a range of 5 cps to 10 cps, or about 6 cps.

The hard shell capsule includes a gelatinizing agent. The gelatinizing agent can include a hydrocolloid. The hydrocolloid can include such items as synthetic gums which are capable of gelling without the addition of alkaline or alkaline earth metal ions. The hydrocolloid can be an anionic polysaccharide, for example gellan gum. Hydrocolloids can also be chosen from natural seaweeds, natural seed gums, natural plant exudates, natural fruit extracts, bio-synthetic gums, and bio-synthetic processed starch. The hydrocolloid can include one or more types chosen from alginates, agar gum, guar gum, locust bean gum (carob), carrageenan (e.g. kappa- and/or iota-carrageenans), tara gum, gum arabic, ghatti gum, Khaya grandifolia gum, tragacanth gum, karaya gum, pectin, arabian (araban), xanthan, gellan, starch, Konjac mannan, galactomannan, funoran, and other exocellular polysaccharides e.g. xanthan, acetan, gellan, welan, rhamsan, furcelleran, succinoglycan, scieroglycan, schizophyflan, tamarind gum, curdlan, pullulan, and dextran. The amount of the hydrocolloid in the hard shell capsule can be 0.01% to 50% by weight of the hard shell capsule, or 0.1% to 30%, or 0.1% to 20%, or 0.1% to 10%, or 0.1% to 2%, or 0.1% to 1.0%. The hard capsule shell can include about 0.01 to about 10% by weight of a gelatinizing agent. The gelatinizing agent can include gellan gum.

The hard shell capsule can further include a gelling promoter. The gelling promoter can be chosen from an ammonium, calcium, magnesium, potassium, or sodium cation, for example, or chosen from a calcium, potassium, or sodium cation. The cation can be provided by use of a water-soluble ammonium salt, calcium salt, magnesium salt, potassium salt, or sodium salt. The gelling promoter can be provided by a water-soluble salt of an organic acid, or a water-soluble salt of an inorganic acid, and in this context can be referred to as a gelling promoter precursor. For example, the gelling promoter precursor can be one or more compounds chosen from the group of ammonium chloride, ammonium acetate, calcium pantothenate, calcium chloride, calcium bromide, calcium lactate, calcium nitrate, magnesium sulfate, potassium acetate, potassium chloride, potassium phosphate, and sodium chloride. Citric acid can be used as a gelling promoter. The amount of gelling promoter precursor can be in a range of about 0.1% to 20% by weight of the hard shell capsule, or 0.5% to 15%, or 0.5% to 10%, for example.

The hard shell capsule can include one or more adjuvants chosen from the group of a plasticizer, a lubricant, a sequestrant, a colorant, a light-shielding agent, and residual moisture (e.g. 1% to 10% of the hard shell capsule weight), and in amounts typically used for such purposes. Hard shell capsules can be purchased commercially, or made by methods known in the art.

The hard capsule shell can be of any suitable size to contain the formulation disposed therein at the desired active strength, e.g. in a range of standard sizes from size 000 to size 5, e.g. size 000, size 00E, size 00, size 0E, size 0, size 1, size 2, size 3, size 4, or size 5, or in a range of size 3 to size 5, or specifically size 3, or specifically size 4, for example.

Once filled, the hard shell optionally can be banded/sealed with a hypromellose solution, e.g. one made from a low viscous grade, e.g. E3 grade which is 3 mPa s viscosity as 2% solution at 20° C. The sealing solution can be applied in any suitable amount, for example in a range of about 0.002 g/cap to 0.02 g/cap, for example. In one aspect, the solution can be applied at a rate that does not affect the dissolution characteristics, but can protect the capsule from leakage with use of liquid contents, or with contents that can become soft or liquid at elevated temperatures encountered in hot zones, eg. 35° C. to 50° C., for example.

The hard shell capsule contains a vitamin D formulation, which can be a 25-hydroxyvitamin D formulation. In various aspects, the dosage form is prepared to administer the 25-hydroxyvitamin D compound orally. In various instances, the 25-hydroxyvitamin D compound comprises 25-hydroxyvitamin D₂ or 25-hydroxyvitamin D₃, or a combination of 25-hydroxyvitamin D₂ and 25-hydroxyvitamin D₃. It is specifically contemplated that in any and every aspect and embodiment of the compositions and methods disclosed herein, the 25-hydroxyvitamin D compound can be 25-hydroxyvitamin D₃. As used herein, the term “25-hydroxyvitamin D compound” refers to one or more of 25-hydroxyvitamin D₃, 25-hydroxyvitamin D₂, 25-hydroxyvitamin D₄, 25-hydroxyvitamin D₅, or 25-hydroxyvitamin D₇, and it is contemplated that in any reference thereto a preferred embodiment is one or more of 25-hydroxyvitamin D₃ and 25-hydroxyvitamin D₂, preferably 25-hydroxyvitamin D₃. Thus, in any and all formulations described herein, it is specifically contemplated that the active can include one or both of 25-hydroxyvitamin D₂ and 25-hydroxyvitamin D₃, particularly 25-hydroxyvitamin D₃.

The hard shell capsule contains a 25-hydroxyvitamin D formulation, a.k.a. a fill formulation, which can take various forms, as described below. Such a formulation can be an extended-release formulation, and further optionally can have delayed release characteristics (e.g., alone, or as a result of use in a gelatinized cellulose ether hard capsule shell according to the disclosure herein).

The 25-hydroxyvitamin D compound, may be administered to the subject by any suitable means. Formulations suitable for oral administration can consist of or include (a) liquid solutions or suspensions, such as an effective amount of the 25-hydroxyvitamin D compound dissolved or suspended in diluents, such as water, saline, milk, oils, or other carriers; (b) as solids or granules; (c) powders; and (d) suitable emulsions. Liquid formulations may include diluents, such as water or alcohols, for example, ethanol, benzyl alcohol, and the polyethylene alcohols, either with or without the addition of a pharmaceutically acceptable surfactant. Capsule forms can contain, for example, carriers, such as oils, waxes, or other lipids, surfactants, lubricants, and inert fillers, such as lactose, sucrose, calcium phosphate, and corn starch. The hard shell capsule can contain a tablet slug, and such tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. The 25-hyroxyvitamin D compound can be dissolved in an alcohol, e.g. ethanol, for distribution in a carrier or excipient.

Oils, which can be used in enteral formulations, include petroleum, animal, vegetable, or synthetic oils. Specific examples of oils include peanut, soybean, sesame, cottonseed, corn, olive, petrolatum, and mineral. Non-digestible oils are contemplated for some embodiments. Ethyl oleate and isopropyl myristate are examples of suitable fatty acid esters.

The 25-hydroxyvitamin D compound can be dispersed in a polymer composition. The 25-hydroxyvitamin D compound can be embedded in a polymer network. The polymer can be water-insoluble, and optionally swellable, for example. The formulation can be a spheronized pellet formulation comprising a 25-hydroxyvitamin D compound and a pharmaceutically acceptable excipient. Such pellets optionally can be enteric coated; in the alternative, the pellets can be disposed in the capsule shell which can be enteric coated. The formulation can include a 25-hydroxyvitamin D compound dispersed in a fatty acid glyceride mixture. The formulation can consist of or include a nano/microparticle formulation comprising a 25-hydroxyvitamin D compound and a pharmaceutically acceptable excipient. The formulation can consist of or include a lipid microparticle formulation comprising a 25-hydroxyvitamin D compound and a pharmaceutically acceptable lipid. The formulation can consist of or include a non-pareil seed formulation comprising a 25-hydroxyvitamin D compound and a pharmaceutically acceptable excipient. The formulation can consist of or include a 25-hydroxyvitamin D compound and a pharmaceutically acceptable excipient selected from one or more excipients in the group of an absorption enhancer, a spheronizing aid, a water insoluble polymer, and a binder. The formulation can consist of or include a spray-congealed lipid vitamin D formulation comprising a 25-hydroxyvitamin D compound, an extended release agent, and a surfactant. In embodiments, the formulation can be an extended release formulation, e.g. for oral use.

As used herein, the formulation comprising the 25-hydroxyvitamin D compound can be a stabilized formulation, wherein “stabilized formulation” refers to a formulation exhibiting a stable in vitro dissolution profile (according to any of the parameters described further herein) and controlled release (e.g., extended release) of a vitamin D compound in vivo, for a time following initial manufacture, e.g. following actual shelf storage or accelerated stability storage conditions. The release of the active ingredient can be measured using a suitable in vitro dissolution method, such as one of the methods already known in the art. In principle, any of the dissolution studies described in the United States Pharmacopeia, USP 43-NF 38 2S, Dissolution <711> physical tests and determinations, United States Pharmacopeial Convention, Inc., Rockville, Md., 2020; European Pharmacopoeia 2.9.3 Dissolution Test for Solid Dosage Forms, or the Japanese Pharmacopoeia 6.10 Dissolution Test, can be used to determine if a formulation is stable. For purposes of the present disclosure, the single medium in vitro dissolution method is United States Pharmacopeia, USP 43-NF 38 2S, Dissolution <711> physical tests and determinations, United States Pharmacopeial Convention, Inc., Rockville, Md., 2020, using Apparatus 2 (paddle method), as described in the embodiments below. In an alternative, dissolution characteristics can be measured using a 2-phase method, such as Method 2 in USP 43-NF 38 2S, Dissolution <711>, using Apparatus 1 or 2, optionally Apparatus 2.

A stabilized formulation according to the disclosure herein, following storage for a period of time, releases an amount of 25-hydroxyvitamin D in in vitro dissolution that does not substantially differ from the dissolution of the same formulation just after manufacturing and prior to storage. For example, in one embodiment, a formulation releases an amount of 25-hydroxyvitamin D during in vitro dissolution after exposure to storage conditions of two months at 25° C. and 60% relative humidity that varies at any given dissolution time point after four hours by 30% or less compared to the amount released at the same dissolution time point during in vitro dissolution conducted prior to exposing the formulation to the storage conditions (i.e., freshly-produced product).

The table below provides examples of advantageous degrees of storage stability contemplated for embodiments of the invention following storage at 25° C. and 60% RH, and alternatively at 40° C. and 75% RH for various times following initial manufacturing, and at various times in during dissolution testing. The degrees of storage stability are expressed in terms of the maximum deviation from nominal active potency, i.e. maximum % change from LC. Alternative embodiments of maximum deviation are also provided.

| | | | | | | | |
|---|---|---|---|---|---|---|---|
| Time (h) | 1 month | 3 mos. | 6 mos. | 9 mos. | 12 mos. | 18 mos. | 24 mos. |

storage at 25° C. and 60% RH

| | | | | | | | |
|---|---|---|---|---|---|---|---|
| 2 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 4 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 6 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 8 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 12 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |

storage at 40° C. and 75% RH

| | | | | | | | |
|---|---|---|---|---|---|---|---|
| 2 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 4 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 6 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 8 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |
| 12 | 30%, or | 30%, or | 30%, or | 30%, or | 30%, or | 30%, or | 30%, or |
| | 25%, or | 25%, or | 25%, or | 25%, or | 25%, or | 25%, or | 25%, or |
| | 20%, or | 20%, or | 20%, or | 20%, or | 20%, or | 20%, or | 20%, or |
| | 15%, or | 15%, or | 15%, or | 15%, or | 15%, or | 15%, or | 15%, or |
| | 10% | 10% | 10% | 10% | 10% | 10% | 10% |

In one type of embodiment, the formulation will have advantageous degrees of stability described in the table immediately above at multiple time points throughout the dissolution testing, e.g. at least at both 2 and 4 hour time points, optionally also at the 6 hour time point, further optionally also at the 8 hour time point, and further optionally also at the 12 hour time point, such that the dissolution profile after storage follows the dissolution profile of fresh product. Alternatively, the formulation will have advantageous degrees of stability described in the table immediately above at least at the 2, 6, and 12 hour time points. Alternatively, the formulation will have advantageous degrees of stability described in the table immediately above at least at the 4, 8, and 12 hour time points. Alternatively, the formulation will have advantageous degrees of stability described in the table immediately above at least at the 2, 4, and 6, hour time points. Alternatively, the formulation will have advantageous degrees of stability described in the table immediately above at least at the 4, 6, 8, and 12 hour time points, or at all times of 4 hours and thereafter.

In any and all of the embodiments described in the table immediately above, it is contemplated that the deviation can be positive (more release) or negative (less release) with respect to the fresh product. In one type of embodiment, it is contemplated that the deviation will be in the negative (less release) direction at multiple time points. Still further, in one type of embodiment it is contemplated that the deviation in dissolution release would have been negative (less release) at multiple time points but for the presence of the stabilizing agent in the formulation.

In various instances, the formulation comprising the 25-hydroxyvitamin D compound comprises a matrix component that releasably binds the vitamin D compound and controllably releases the vitamin D compound (e.g., a lipophilic matrix), and a stabilizer (e.g. a cellulosic compound). In various instances, the stabilizing agent is a cellulosic compound. As used herein, the term “cellulosic compound” can include cellulose (C₆H₁₀O₅)ₙ or a derivative of cellulose, unless specified otherwise. In various aspects, the cellulosic compound is a cellulose ether. A “cellulose ether” is a cellulose derivative that has been chemically modified to result in partial or complete etherification of the hydroxyl groups in the cellulose molecule. Examples of cellulose derivatives which can be used as stabilizing agents include, but are not limited to, celluloronic acid, carboxy methyl cellulose, ethyl cellulose, hydroxyethyl cellulose, hydroxyl propyl cellulose, hydroxyl propyl methylcellulose, methyl cellulose, polyanionic cellulose, and combinations thereof, for example. Different grades of each cellulosic compound or stabilizing agent, corresponding to variations in, e.g., molecular weight, viscosity, solubility, and hydration, are also encompassed by the terms.

In one embodiment, a stabilized formulation comprises one or both of 25-hydroxyvitamin D₂ and 25-hydroxyvitamin D₃, a wax matrix, and a cellulosic compound. In one aspect, a stabilized formulation comprises one or both of 25-hydroxyvitamin D₂ and 25-hydroxyvitamin D₃, a wax matrix, and a cellulosic stabilizing agent. In another aspect, the formulation comprises one or both of 25-hydroxyvitamin D₂ and 25-hydroxyvitamin D₃, a wax matrix, and an effective amount of a cellulosic compound to provide an advantageous degree of stability as described herein, e.g. with respect to the table immediately above or consistent with any of the Examples described below. For example, the amount can be effective to provide a difference of 30% or less between the amount of active released during in vitro dissolution after exposure to storage conditions of at least one month at 25° C. and 60% relative humidity at a dissolution time point and the amount released at the same dissolution time point during in vitro dissolution conducted prior to exposing the formulation to the storage conditions, while a comparative formulation lacking the stabilizing agent would result in a greater difference in dissolution release following the same storage conditions.

In one aspect, the formulation is an improved formulation for controlled release of a vitamin D compound in the gastrointestinal tract of a subject which ingests the formulation. In one embodiment, the improvement comprises admixing a cellulosic stabilizing agent into a formulation for controlled release of a vitamin D compound in the gastrointestinal tract of a subject which ingests the formulation. In another embodiment, the improvement comprises an effective amount of a cellulosic compound admixed into a formulation for controlled release of a vitamin D compound in the gastrointestinal tract of a subject which ingests the formulation to provide an advantageous degree of stability as described herein, e.g. with respect to the table immediately above or consistent with any of the examples described below. For example, the amount can be effective to provide a difference of 30% or less between the amount of active released during in vitro dissolution after exposure to storage conditions of at least one month at 25° C. and 60% relative humidity at a dissolution time point and the amount released at the same dissolution time point during in vitro dissolution conducted prior to exposing the formulation to the storage conditions, while a comparative formulation lacking the stabilizing agent would result in a greater difference in dissolution release following the same storage conditions.

The stabilizing agents can include cellulose compounds. Examples of cellulose compounds and stabilizing agents for use in the stabilized formulations of the disclosure can include, but are not limited to, celluloronic acid, carboxy methyl cellulose, ethyl cellulose, hydroxyl ethyl cellulose, hydroxyl propyl cellulose, hydroxyl propyl methyl cellulose, methylcellulose, polyanionic cellulose, and combinations thereof. Also contemplated are one or more of poloxamers (e.g., poloxamer 407), poly (ethylene oxide) polymers (e.g., Dow's POLYOX polymers), povidones, and fumed silicas (e.g., AEROSIL 200, Evonik Industries AG, Essen, Germany) The stabilizer, e.g. a cellulosic compound, preferably is present in an amount of at least about 5% of the formulation, based on the total weight of the formulation excluding any additional coatings or shells (wt %). For example, the cellulosic compound can be present in an amount of at least 5 wt % of the formulation, or at least 10 wt % of the formulation, or at least 15 wt % of the formulation, or greater than 5 wt % of the formulation, or greater than 10 wt % of the formulation, or greater than 15 wt % of the formulation. Suitable ranges include 5 wt % to 30 wt %, 10 wt % to 20 wt %, 10 wt % to 15 wt %, 5 wt % to 15 wt %, and 7.5 wt % to 12.5 wt. %. Examples include about 5 wt %, about 6 wt %, about 7 wt %, about 8 wt %, about 9 wt %, about 10 wt %, about 11 wt %, about 12 wt %, about 13 wt %, about 14 wt %, and about 15 wt %. It will be understood that the stabilizing agent referred to herein is an agent that stabilizes the dissolution release profile (and thus also the in vivo release profile) against substantial change over time during storage conditions, e.g. typical shelf storage conditions. Other agents which are known in the art as preservatives for preventing degradation of the active component itself are not intended to be encompassed within the terms “stabilizing agent” and “stabilizer” although such preservatives are also contemplated for use in the formulations of the present invention.

In one class of embodiment, the cellulosic compound is a cellulose ether. Examples of cellulose ethers include, but are not limited to, methylcellulose, hydroxyl propyl methylcellulose, hydroxyl ethyl methylcellulose, hydroxyl ethyl cellulose, hydroxyl propyl cellulose, and combinations thereof.

Hydroxypropyl methylcellulose (HPMC, hypromellose) is particularly contemplated. The HPMC can be characterized by one or more of the following features, which are specifically contemplated individually and in combinations. The % methyoxyl component in the HPMC can be in a range of 19 to 24. The % hydroxypropyl component can be in a range of 7 to 12. The apparent viscosity (2% solution in water at 20° C.) can be at least 50,000 cP, or at least 80,000 cP, or in a range of about 80 to 120,000 cP, or 3000 to 120,000 cP, or 11,000 to 120,000 cP, or 80,000 to 120,000 cP. Particularly, the apparent viscosity (2% solution in water at 20° C.) can be in a range of 80,000 to 120,000 cP. The pH (1% solution in water) can be in a range of 5.5 to 8.0. For example, a suitable hydroxyl propyl methylcellulose having all of the foregoing properties, including an apparent viscosity (2% solution in water at 20° C.) in a range of 80,000 to 120,000 cP, is METHOCEL K100M CR (Dow Wolff Cellulosics, Midland, Mich.).

In one type of embodiment, the cellulosic compound will be insoluble in the matrix formulation at the melt point of the primary components of the matrix, e.g., at 65° C. or in a range of 60° C. to 75° C.

In one type of embodiment, the cellulosic compound will be hydrophilic. The stabilized wax matrix formulation (e.g., Rayaldee®-type fill formulation) can have the following composition filled into a hard shell capsule according to the disclosure herein, instead of filled into soft OptiShell® plant polysaccharide shells as with Rayaldee® (calcifediol) extended-release capsules: calcifediol 0.02% of capsule fill by weight, paraffin 20.0% of capsule fill by weight, mineral oil 35.34% of capsule fill by weight, Hypromellose 10.0% of capsule fill by weight, mono- and di-glycerides 22.56% of capsule fill by weight, lauroyl polyoxylglycerides 9.75% of capsule fill by weight, dehydrated alcohol 2.32% of capsule fill by weight, and BHT 0.02% of capsule fill by weight.

The pharmaceutical formulations according to the disclosure comprising one of more of 25-hydroxyvitamin D₂ and 25-hydroxyvitamin D₃ and a cellulosic compound have improved stability compared to formulations lacking a cellulosic compound. In one embodiment, a stabilized formulation according to the disclosure comprises a mixture of an active-loaded lipophilic matrix comprising one or both of 25-hydroxyvitamin D₂ and 25-hydroxyvitamin D₃ and a cellulosic stabilizing agent, wherein the formulation releases an amount of 25-hydroxyvitamin D during in vitro dissolution after exposure to storage conditions of at least one month at 25° C. and 60% relative humidity that varies at any given dissolution time point by 30% or less compared to the amount released at the same dissolution time point during in vitro dissolution conducted on freshly-made product.

Formulations that are not stabilized exhibit changes in the amount of active ingredient released after the composition is stored for a period of time. An unstabilized formulation releases an amount of 25-hydroxyvitamin D following exposure to storage conditions that can vary at a given dissolution time point, for example by more than 30% compared to the amount released at the same dissolution time point during in vitro dissolution conducted on freshly-made product. The changes may be an increase or decrease in the dissolution rate at a given time point, and such changes produce a dissolution profile whose curve is distinct from the shape of the initial dissolution profile. An unstabilized formulation also exhibits different in vivo effects compared to a stabilized formulation according to the disclosure, following storage as described herein, e.g. following 3 months or more of storage at 25° C. and 60% RH. A stabilized formulation demonstrates different clinical pharmacokinetic parameters, such as improved bioavailability, compared to an unstabilized formulation, following storage as described herein, e.g. following 3 months or more of storage at 25° C. and 60% RH. A stabilized formulation according to the disclosure can have a base formulation which is storage unstable, combined with a stabilizing agent which renders the formulation storage stable as described herein.

The matrix that releasably binds and controllably releases the active component can be, for example, a lipophilic matrix, including a wax matrix. A wax matrix can provide a formulation which is solid or semi-solid at room temperature and solid, semi-solid, or liquid at body temperature, preferably semi-solid or liquid at body temperature. In one aspect, the wax matrix comprises a controlled release agent, an emulsifier, and an absorption enhancer.

Examples of controlled release agents suitable for use include, but are not limited to, waxes, including synthetic waxes, microcrystalline wax, paraffin wax, carnauba wax, and beeswax; polyethoxylated castor oil derivatives, hydrogenated vegetable oils, glyceryl mono-, di- or tribehenates; long-chain alcohols, such as stearyl alcohol, cetyl alcohol, and polyethylene glycol; and mixtures of any of the foregoing. Non-digestible waxy substances, such as hard paraffin wax, are preferred.

The controlled release agent can be present in an amount of at least 5 wt % of the stabilized matrix formulation, or greater than about 5 wt % of the formulation. For example, depending on the controlled release agent used, the controlled release agent can comprise at least 5 wt % of the formulation or at least 10 wt % of the formulation, or at least 15 wt % of the formulation, or at least 20 wt % of the formulation, or at least 25 wt % of the formulation, or greater than 5 wt % of the formulation, or greater than 10 wt % of the formulation, or greater than 15 wt % of the formulation, or greater than 20 wt % of the formulation, and or greater than 25 wt % of the formulation. The controlled release agent can be present in an amount 50 wt % or less, 40 wt % or less, 35 wt % or less, or 30 wt % or less. Suitable ranges include 5 wt % to 40 wt %, 10 wt % to 30 wt % and 15 wt % to 25 wt %. Examples include about 15 wt %, about 16 wt %, about 17 wt %, about 18 wt %, about 19 wt %, about 20 wt %, about 21 wt %, about 22 wt %, about 23 wt %, about 24 wt %, and about 25 wt %.

Examples of emulsifiers suitable for use in the stabilized matrix formulation include, but are not limited to, lipophilic agents having an HLB of less than 7, such as mixed fatty acid monoglycerides; mixed fatty acid diglycerides; mixtures of fatty acid mono- and di-glycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan esters including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan esters including sorbitan monostearate, sorbitan sesquioleate; fatty acids and their soaps including stearic acid, palmitic acid, and oleic acid; and mixtures thereof.

A preferred lipoidic agent for use in the stabilized matrix formulation is selected from glycerides and derivatives thereof. Preferred glycerides are selected from the group consisting of medium or long chain glycerides, caprylocaproyl macrogolglycerides, and mixtures thereof.

Preferred medium chain glycerides include, but are not limited to, medium chain monoglycerides, medium chain diglycerides, caprylic/capric triglyceride, glyceryl monolaurate, glyceryl monostearate, caprylic/capric glycerides, glycerylmonocaprylate, glyceryl monodicaprylate, caprylic/capric linoleic triglyceride, and caprylic/capric/succinic triglyceride.

Monoglycerides having a low melting point are preferred for making the stabilized matrix formulation. Preferred monoglycerides include but are not limited to, glyceryl monostearate, glyceryl monopalmitate, glyceryl monooleate, glyceryl monocaprylate, glyceryl monocaprate, glyceryl monolaurate, etc., preferably glycerol monostearate (GMS). GMS is a natural emulsifying agent. It is oil soluble, but poorly soluble in water. GMS has an HLB value of 3.8. The lipophilic emulsifier can be present in an amount in a range of about 10 wt % to about 40 wt %, or about 20 wt % to about 25 wt %, for example. Other examples include about 20 wt %, about 21 wt %, about 22 wt %, about 23 wt %, about 24 wt %, and about 25 wt %.

Examples of suitable absorption enhancers for use in the stabilized matrix formulation include, but are not limited to, caprylocaproyl macrogolglycerides such as polyethylene glycosylated glycerides, also known as polyglycolized glycerides or PEGylated glycerides. PEGylated glycerides which may be employed in the composition include, but are not limited to, mixtures of monoglycerides, diglycerides, and triglycerides and monoesters and diesters of polyethylene glycol, polyethylene glycosylated almond glycerides, polyethylene glycosylated corn glycerides, and polyethylene glycosylated caprylic/capric triglyceride. The absorption enhancer can have an HLB value from 13 to 18, or from 13 to 15.

One preferred absorption enhancer is known under the trade name GELUCIRE (Gattefossé Corporation, Paramus, N.J., USA). GELUCIRE is a well-known excipient which is a family of fatty acid esters of glycerol and PEG esters, also known as polyglycolized glycerides. GELUCIRE is used in various applications including preparing sustained release pharmaceutical compositions. GELUCIRE compounds are inert, semi-solid waxy materials which are amphiphilic and are available with varying physical characteristics such as melting point, HLB, and solubilities in various solvents. They are surface active in nature and disperse or solubilize in aqueous media forming micelles, microscopic globules or vesicles. They are identified by their melting point/HLB value. The melting point is expressed in degrees Celsius. One or a mixture of different grades of GELUCIRE excipient may be chosen to achieve the desired characteristics of melting point and/or HLB value. A preferred GELUCIRE composition is GELUCIRE 44/14, a mixture of lauroyl macrogolglycerides and lauroyl polyoxylglycerides that has a melting point of 44° C. and a HLB of 14. The absorption enhancer can be present in an amount of about 5 wt % to about 20 wt %, or about 8 wt % to about 15 wt %, for example. Other examples include about 8 wt %, about 9 wt %, about 10 wt %, about 11, wt % about 12 wt %, about 13 wt %, about 14 wt %, and about 15 wt %.

The low melting points of the wax matrix provide a means of incorporating the pharmaceutically active ingredients, e.g. the vitamin D compound such as 25-hydroxyvitamin D₂, 25-hydroxyvitamin D₃, or both, at temperatures from about 0° C. to about 50° C. above the melting point of the wax matrix and then filling the melt (solution and/or dispersion) in suitable capsules. The capsules can be of any variety that is compatible with the temperature of the melt fill, including soft or hard gelatin capsules, and animal or vegetable gelatin capsules. The melt solidifies inside the capsules upon cooling to room temperature.

In one aspect, the stabilized matrix formulation may further comprise an oily vehicle for the 25-hydroxyvitamin D₂ and/or 25-hydroxyvitamin D₃. Any pharmaceutically-acceptable oil can be used. Examples include animal (e.g., fish), vegetable (e.g., soybean), and mineral oils. The oil preferably will readily dissolve the 25-hydroxyvitamin D compound used. Preferred oily vehicles include non-digestible oils, such as mineral oils, particularly liquid paraffins, and squalene. The oily vehicle can be present at a concentration in a range about 10 wt % to about 50 wt % of the formulation, or about 15 wt % to about 45 wt %, or about 20 wt % to about 40 wt %, or about 30 wt % to about 40 wt %, for example. In one type of embodiment, a suitable liquid paraffin can be characterized by one or more of the following parameters: specific gravity about 0.88 to 0.89; kinematic viscosity (40° C.) about 64 cSt to about 70 cSt; molecular weight 424; % paraffinic hydrocarbons about 59; and pour point −24° C. The ratio between the wax matrix and the oily vehicle can be optimized in order to achieve the desired rate of release of the vitamin D compound. Thus, if a heavier oil component is used, relatively less of the wax matrix can be used, and if a lighter oil component is used, then relatively more wax matrix can be used.

The stabilized controlled release compositions in accordance with the disclosure preferably are designed to contain concentrations of 25-hydroxyvitamin D₂ and/or 25-hydroxyvitamin D₃ of 1 to 1000 μg per unit dose, for example, and are prepared in such a manner as to effect controlled or substantially constant release of the 25-hydroxyvitamin D₂/25-hydroxyvitamin D₃, optionally into the ileum of the gastrointestinal tract, of humans or animals over an extended period of time. Example dosages include 1 μg to 1000 μg per unit dose, 1 μg to 600 μg, 1 μg to 500 μg, 1 μg to 450 μg, 1 μg to 400 μg, 1 μg to 200 μg, 1 μg to 100 μg, 5 μg to 90 μg, 30 μg to 80 μg, 20 μg to 60 μg, 30 μg to 60 μg, 35 μg to 50 μg, 5 μg to 50 μg, and 10 μg to 25 μg, for example 20 μg, 25 μg, 30 μg, 40 μg, 50 μg, 60 μg, 70 μg, 80 μg, 90 μg, and 100 μg.

One type of hard capsule formulation has release-modifying agents including a lipophilic (optionally waxy) fill, emulsifiers, and an absorption enhancer, e.g. same or similar to the wax-based matrix formulations described above, or omitting wax and including higher concentrations of other lipophilic release agents instead. The matrix can be solid or semi-solid at both room temperature and at the normal temperature of the human body. It starts releasing slowly and in a substantially constant fashion, controlling release of the active for a period of at least 4 hours, or at least 8 hours, or at least 10 hours, or at least 12 hours, optionally in a range of 4 to 24 hours, or 6 to 20 hours, or 8 to 18 hours, or 10 to 16, hours, or about 12 hours. The release mechanism can be governed by mechanical erosion and/or gradual disintegration, into the contents of the lumen of the lower small intestine and/or colon, for example.

The composition comprising the 25-hydroxyvitamin D compound in the gelatinized hypromellose shell can be any one described herein, for example a solid or semi-solid composition, optionally a wax matrix. The amount of wax can be about 20 wt. % to about 36 wt. % based on the weight of the solid or semi-solid composition. The wax of the wax matrix can include a non-digestible wax, optionally paraffin wax. The composition comprising the 25-hydroxyvitamin D compound can further include an oily vehicle, optionally in an amount of about 25 wt. % to about 41 wt. % based on the weight of the solid or semi-solid composition. The oily vehicle can include or consist of a non-digestible oil, optionally mineral oil. The composition including the 25-hydroxyvitamin D compound can further include stabilizing agent, optionally in an amount in a range of about 2 wt. % to about 18 wt. % based on the weight of the solid or semi-solid composition. The stabilizing agent can include a cellulose ether, for example hydroxypropyl methylcellulose. The composition comprising the 25-hydroxyvitamin D compound can further include an emulsifier, e.g. in an amount in a range of about 10 wt. % to about 26 wt. % based on the weight of the solid or semi-solid composition. The emulsifier can include mono- and diglyceryl esters of long chain, saturated and unsaturated fatty acids, for example. The composition comprising the 25-hydroxyvitamin D compound can further include an absorption enhancer, optionally in an amount in a range of about 3 wt. % to about 17 wt. % based on the weight of the solid or semi-solid composition. The absorption enhancer can include or consist of fatty acid esters of glycerol and PEG esters, optionally lauroyl polyoxylglycerides. The composition comprising the 25-hydroxyvitamin D compound can further include a solvent for the 25-hydroxyvitamin D, optionally in an amount in a range of about 0.2 wt. % to about 6 wt. % based on the weight of the solid or semi-solid composition. The solvent can include or consist of an alcohol, optionally ethanol. The hard capsule dosage form can include the 25-hydroxyvitamin D compound in an amount in a range of about 0.1 μg to about 2 mg, for example. The 25-hydroxyvitamin D compound can include or consist of 25-hydroxyvitamin D₃. The dosage form can include 6 μg to 500 μg bioavailable 25-hydroxyvitamin D, for example. The hard capsule dosage form can be used to treat secondary hyperparathyroidism in a patient having stage 3, 4 or 5 Chronic Kidney Disease. The formulation type of this paragraph is contemplated for use also in a non-gelatinized hard capsule shell, as are the formulations of Example 1 (0%, 10%, 20%, 30%, and 40% paraffin wax types), Example 2 (Test 3 and Test 4 types).

Thus, another aspect of the disclosure is a composition comprising a 25-hydroxyvitamin D compound described herein and contained in a non-gelatinized hypromellose shell. For example, the composition can be a solid or semi-solid composition, optionally a wax matrix. The amount of wax can be about 20 wt. % to about 36 wt. % based on the weight of the solid or semi-solid composition. The wax of the wax matrix can include a non-digestible wax, optionally paraffin wax. The composition comprising the 25-hydroxyvitamin D compound can further include an oily vehicle, optionally in an amount of about 25 wt. % to about 41 wt. % based on the weight of the solid or semi-solid composition. The oily vehicle can include or consist of a non-digestible oil, optionally mineral oil. The composition including the 25-hydroxyvitamin D compound can further include stabilizing agent, optionally in an amount in a range of about 2 wt. % to about 18 wt. % based on the weight of the solid or semi-solid composition. The stabilizing agent can include a cellulose ether, for example hydroxypropyl methylcellulose. The composition comprising the 25-hydroxyvitamin D compound can further include an emulsifier, e.g. in an amount in a range of about 10 wt. % to about 26 wt. % based on the weight of the solid or semi-solid composition. The emulsifier can include mono- and diglyceryl esters of long chain, saturated and unsaturated fatty acids, for example. The composition comprising the 25-hydroxyvitamin D compound can further include an absorption enhancer, optionally in an amount in a range of about 3 wt. % to about 17 wt. % based on the weight of the solid or semi-solid composition. The absorption enhancer can include or consist of fatty acid esters of glycerol and PEG esters, optionally lauroyl polyoxylglycerides. The composition comprising the 25-hydroxyvitamin D compound can further include a solvent for the 25-hydroxyvitamin D, optionally in an amount in a range of about 0.2 wt. % to about 6 wt. % based on the weight of the solid or semi-solid composition. The solvent can include or consist of an alcohol, optionally ethanol. The hard capsule dosage form can include the 25-hydroxyvitamin D compound in an amount in a range of about 0.1 μg to about 2 mg, for example. The 25-hydroxyvitamin D compound can include or consist of 25-hydroxyvitamin D₃. The dosage form can include 6 μg to 500 μg bioavailable 25-hydroxyvitamin D, for example. The hard capsule dosage form can be used to treat secondary hyperparathyroidism in a patient having stage 3, 4 or 5 Chronic Kidney Disease.

The following formulations can be disposed in a non-gelatinized HPMC hard capsule.

| | 0% | 10% | 20% | 30% | 40% | | |
|---|---|---|---|---|---|---|---|
| | P. wax | P. wax | P. wax | P. wax | P. wax | Size 3 | Size 4 |
| Material | % Cap | % Cap | % Cap | % Cap | % Cap | capsule | capsule |
| calcifediol | 0.0176% | 0.0176% | 0.0176% | 0.0176% | 0.0176% | 0.0176% | 0.0194% |
| paraffin | 0.00% | 10.00% | 20.00% | 30.00% | 40.00% | 28.00% | 19.95% |
| mineral oil | 55.34% | 45.34% | 35.34% | 25.34% | 15.34% | 27.39% | 35.26% |
| hypromellose K100 | 10.00% | 10.00% | 10.00% | 10.00% | 10.00% | 10.00% | 9.98% |
| mono-and di- | 22.55% | 22.55% | 22.55% | 22.55% | 22.55% | 20.50% | 22.50% |
| glycerides | | | | | | | |
| lauroyl | 9.75% | 9.75% | 9.75% | 9.75% | 9.75% | 11.75% | 9.73% |
| polyoxylglycerides | | | | | | | |
| dehydrated ethanol | 2.32% | 2.32% | 2.32% | 2.32% | 2.32% | 2.32% | 2.54% |
| BHT | 0.02% | 0.02% | 0.02% | 0.02% | 0.02% | 0.02% | 0.02% |
| Total | 100.00% | 100.00% | 100.00% | 100.00% | 100.00% | 100.00% | 100.00% |

The calcifediol hard capsule formulation can be prepared by any suitable method, including filling a capsule shell with a flowable material, or filling a capsule shell with a mass or slug of solid or semi-solid material, or enrobing or coating a solid or semisolid mass with a shell composition, for example. The size of the hard capsule can be adjusted depending upon the particular fill ratios of the paraffin and the other excipients, e.g. from size 3 to size 4, to further control the release of the drug.

Provided in the table below are example HPMC hard capsule formulations of 25-hydroxyvitamin D with varied percentages of excipients (all percentages by weight, based on the weight of the fill material in the capsule).

| | ~0% P. wax | 0% P. wax | 0% P. wax | 10% P. wax | 20% P. wax | 30% P. wax | 40% P. wax |
|---|---|---|---|---|---|---|---|
| Material | % Cap | % Cap | % Cap | % Cap | % Cap | % Cap | % Cap |
| calcifediol | 0.0176% | 0.0176% | 0.0176% | 0.0176% | 0.0176% | 0.0176% | 0.0176% |
| paraffin | 0-2% | 0.00% | 0.00% | 10.00% | 20.00% | 30.00% | 40.00% |
| mineral oil | 45.34% | 35.34% | 55.34% | 45.34% | 35.34% | 25.34% | 15.34% |
| hypromellose K100 | 10-15% | 10.00% | 10.00% | 10.00% | 10.00% | 10.00% | 10.00% |
| mono-and di-glycerides | 22-28% | 41.5% | 22.55% | 22.55% | 22.55% | 22.55% | 22.55% |
| lauroyl polyoxylglycerides | 14.75% | 10.75% | 9.75% | 9.75% | 9.75% | 9.75% | 9.75% |
| dehydrated ethanol | 2.32% | 2.32% | 2.32% | 2.32% | 2.32% | 2.32% | 2.32% |
| BHT | 0.02% | 0.02% | 0.02% | 0.02% | 0.02% | 0.02% | 0.02% |
| Total | 100.00% | 100.00% | 100.00% | 100.00% | 100.00% | 100.00% | 100.00% |

A design of experiments (DOE) study was carried out, varying paraffin wax between 20-40% by weight of the formulation (not including the shell material), the lauroyl polyoxylglycerides from 4.75-14.75%, the mono- and di-glycerides between 22.5-12.5% and HPMC between 6-14%. The mineral oil was kept constant in all formulations as 30%. From this DOE, it was found that to achieve a slower in vitro release profile than the Rayaldee® (calcifediol) extended-release capsules, the paraffin wax percentages can be greater than >35% and the lauroyl polyoxylglycerides around 4.75%.

The table below provides examples of additional wax-based hard capsule formulations, a Rayaldee-®-type soft capsule formulation (Reference) with a vegetable-based capsule shell, and modified wax-based soft vegetable-based capsule formulations. The soft capsules can be OptiShell® vegetable-based capsules, containing modified starch and carrageenan, for example.

| | | | Modified | Modified | | |
|---|---|---|---|---|---|---|
| | | | wax- | wax- | | |
| | | Rayaldee ®- | based | based | HPMC | HPMC |
| | | type soft | soft | soft | Hard | Hard |
| | | capsule | capsule | capsule | capsule | capsule |
| | | (Reference) | (Slow) | (Fast) | (size 3) | (size 4) |
| Excipient | Function | Reference | Test 2 | Test 1 | Test 3 | Test 4 |
| calcifediol | 25- | 0.0176% | 0.0176% | 0.0176% | 0.0176% | 0.0194% |
| | hydroxyvitamin | | | | | |
| | D active | | | | | |
| paraffin wax | control release | 20.00% | 39.00% | 5.00% | 28.00% | 19.95% |
| | agent | | | | | |
| mineral oil | carrier | 35.34% | 30.34% | 45.34% | 27.39% | 35.26% |
| hypromellose | stabilizer | 10.00% | 10.00% | 10.00% | 10.00% | 9.98% |
| mono & | emulsifier | 22.55% | 13.55% | 22.55% | 20.50% | 22.50% |
| diglycerides | | | | | | |
| lauroyl | absorption | 9.75% | 4.75% | 14.75% | 11.75% | 9.73% |
| polyoxyl | enhancer | | | | | |
| glycerides | | | | | | |
| dehydrated | solvent | 2.32% | 2.32% | 2.32% | 2.32% | 2.54% |
| ethanol | | | | | | |
| BHT | antioxidant | 0.02% | 0.02% | 0.02% | 0.02% | 0.02% |
| Total | — | 100.00% | 100.00% | 100.00% | 100.00% | 100.00% |

Described in the table below is another hard capsule formulation of 25-hydroxyvitamin D, with a gelatinized HPMC capsule shell. Gellan gum is a hydrophilic polymer and has similar properties to carrageenan used in the vegetable capsule shells of the Reference soft capsule formulation described above. The gelatinized HPMC capsule has a slower rupture/disintegration time in the stomach than non-gelatinized HPMC capsules.

| Fill Material | % of fill by weight | mg/Cap |
|---|---|---|
| calcifediol | 0.0194% | 0.03 |
| paraffin | 27.95% | 43.32 |
| mineral oil | 32.26% | 50 |
| hypromellose k100 | 9.98% | 15.47 |
| mono-and di-glycerides | 17.5% | 27.13 |
| lauroyl polyoxylglycerides | 9.73% | 15.08 |
| dehydrated ethanol | 2.54% | 3.94 |
| BHT | 0.02% | 0.03 |
| total | 100% | 155 |
| Shell Material | % of shell by weight | mg/Cap |
| hypromellose | qsp100 | 35.283 |
| gellan gum | 5 | 1.9 |
| titanium dioxide | 2 | 0.76 |
| Organic colorant | 0.15 | 0.057 |
| Total | 100 | 38 |

The composition can be filled in size 4 gelatinized HPMC capsule shells, e.g. HPMC capsules containing gellan gum.

Thus, another aspect of the disclosure herein is a gelatinized HPMC hard capsule formulation of 25-hydroxyvitamin D. The formulation can comprise 0.1 μg to about 2 mg of a 25-hydroxyvitamin D compound per unit dose, optionally 25-hydroxyvitamin D₂ and/or 25-hydroxyvitamin D₃. The amount of 25-hydroxyvitamin D compound can further be in a range of about 1 μg to about 1 mg, or about 10 μg to about 900 μg, or about 20 μg to about 600 μg, or about 30 μg to about 300 μg, or about 60 μg to about 300 μg, for example about 20 μg, or about 25 μg, or about 30 μg, or about 40 μg, or about 50 μg, or about 60 μg, or about 70 μg, or about 80 μg, or about 200 μg, or about 300 μg, or about 600 μg, or about 900 μg. The formulation can include about 20 wt. % to about 36 wt. % of a wax, optionally a non-digestible wax, e.g. paraffin wax, based on the total weight of the fill material in the hard capsule shell. The amount of wax can further be in a range of about 22 wt. % to about 34 wt. %, or about 24 wt. % to about 32 wt. %, or about 26 wt. % to about 30 wt. %, for example about 25 wt. %, about 26 wt. %, about 27 wt. %, about 28 wt. %, about 29 wt. %, about 30 wt. %, about 31 wt. %, about 32 wt. %, or about 33 wt. %. The formulation can include about 25 wt. % to about 41 wt. % of an oily vehicle, optionally one described above, e.g. a non-digestible oil, e.g. mineral oil, based on the total weight of the fill material in the hard capsule shell. The amount of oily vehicle can further be in a range of about 27 wt. % to about 39 wt. %, or about 29 wt. % to about 37 wt. %, or about 31 wt. % to about 35 wt. %, for example about 29 wt. %, about 30 wt. %, about 31 wt. %, about 32 wt. %, about 33 wt. %, about 34 wt. %, about 35 wt. %, about 36 wt. %, or about 37 wt. %. The formulation can include about 2 wt. % to about 18 wt. % of a stabilizing agent, optionally one described above, e.g. a cellulose ether, e.g. hypromellose, based on the total weight of the fill material in the hard capsule shell. The amount of stabilizing agent can further be in a range of about 4 wt. % to about 16 wt. %, or about 6 wt. % to about 14 wt. %, or about 8 wt. % to about 12 wt. %, for example about 5 wt. %, about 6 wt. %, about 7 wt. %, about 8 wt. %, about 9 wt. %, about 10 wt. %, about 11 wt. %, about 12 wt. %, or about 13 wt. %. The formulation can include about 10 wt. % to about 26 wt. % of an emulsifier, optionally one described above, e.g. mixtures including mono- and diglyceryl esters of long chain, saturated and unsaturated fatty acids, e.g. mono- and di-glycerides NF, based on the total weight of the fill material in the hard capsule shell. The amount of emulsifier can further be in a range of about 12 wt. % to about 24 wt. %, or about 14 wt. % to about 22 wt. %, or about 16 wt. % to about 20 wt. %, for example about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, or about 23 wt. %. The formulation can include about 3 wt. % to about 17 wt. % of an absorption enhancer, optionally one described above, e.g. fatty acid esters of glycerol and PEG esters, e.g. lauroyl polyoxylglycerides (44/14) based on the total weight of the fill material in the hard capsule shell. The amount of absorption enhancer can further be in a range of about 5 wt. % to about 15 wt. %, or about 7 wt. % to about 13 wt. %, or about 9 wt. % to about 11 wt. %, for example about 6 wt. %, about 7 wt. %, about 8 wt. %, about 9 wt. %, about 10 wt. %, about 11 wt. %, about 12 wt. %, or about 13 wt. %. The 25-hydroxyvitamin D active can be dissolved in an alcohol carrier, e.g. ethanol, which is present in the formulation in an amount of about 0.2 wt. % to about 6 wt. %, or about 0.5 wt. % to about 5 wt. %, or about 1 wt. % to about 4 wt. %, or about 2 wt. % to about 4 wt. %, for example about 1.5 wt. %, or about 2.0 wt. % or about 2.5 wt. %, or about 3 wt. %, or about 3.5 wt. %, or about 4 wt. %. The formulation can include a small amount of a preservative, e.g. an antioxidant, e.g. BHT, e.g. in a range of about 0.005 wt. % to about 1 wt. %, or about 0.01 wt. % to about 0.05 wt. %, e.g. about 0.02 wt. %

Variations of the foregoing type of dosage form can have the following characteristics:

| Fill Material | % of fill by weight |
|---|---|
| calcifediol | about 0.01% to 0.03% |
| paraffin | about 25% to 30% |
| mineral oil | about 30% to 35% |
| hypromellose k100 | about 7% to 13% |
| mono-and di-glycerides | about 14.5% to 20.5% |
| lauroyl polyoxylglycerides | about 7% to 13% |
| dehydrated ethanol | about 2% to 4% |
| BHT | about 0.05% to 0.05% |
| Shell Material | % of shell by weight |
| hypromellose | qsp100 |
| gellan gum | about 1% to 10% |
| titanium dioxide | about 0.01% to 4% |

The amount of fill material can be less than 170 mg and fit in a standard size 4 hard shell capsule, for example in a range of about 150 mg to 160 mg, or 155 mg.

In an alternative hard capsule formulation type, the wax can be omitted and, for example, the concentration of emulsifier and/or absorption enhancer increased. The formulation can comprise 0.1 μg to about 2 mg of a 25-hydroxyvitamin D compound per unit dose, optionally 25-hydroxyvitamin D₂ and/or 25-hydroxyvitamin D₃. The amount of 25-hydroxyvitamin D compound can further be in a range of about 1 μg to about 1 mg, or about 10 μg to about 900 μg, or about 20 μg to about 600 μg, or about 30 μg to about 300 μg, or about 60 μg to about 300 μg, for example about 20 μg, or about 25 μg, or about 30 μg, or about 40 μg, or about 50 μg, or about 60 μg, or about 70 μg, or about 80 μg, or about 200 μg, or about 300 μg, or about 600 μg, or about 900 μg. The formulation can include about 25 wt. % to about 50 wt. % of an oily vehicle, optionally one described above, e.g. a non-digestible oil, e.g. mineral oil, based on the total weight of the fill material in the hard capsule shell. The amount of oily vehicle can further be in a range of about 25 wt. % to about 45 wt. %, or 27 wt. % to about 45 wt. %, or 27 wt. % to about 39 wt. %, or about 29 wt. % to about 37 wt. %, or about 31 wt. % to about 35 wt. %, for example about 30 wt. %, about 32 wt. %, about 34 wt. %, about 36 wt. %, about 38 wt. %, about 40 wt. %, about 42 wt. %, about 44 wt. %, or about 46 wt. %. The formulation can include about 2 wt. % to about 20 wt. % of a stabilizing agent, optionally one described above, e.g. a cellulose ether, e.g. hypromellose, based on the total weight of the fill material in the hard capsule shell. The amount of stabilizing agent can further be in a range of about 4 wt. % to about 16 wt. %, or about 6 wt. % to about 14 wt. %, or about 8 wt. % to about 12 wt. %, for example about 5 wt. %, about 6 wt. %, about 7 wt. %, about 8 wt. %, about 9 wt. %, about 10 wt. %, about 11 wt. %, about 12 wt. %, or about 14 wt. %. The formulation can include about 15 wt. % to about 45 wt. % of an emulsifier, optionally one described above, e.g. mixtures including mono- and diglyceryl esters of long chain, saturated and unsaturated fatty acids, e.g. mono- and di-glycerides NF, based on the total weight of the fill material in the hard capsule shell. The amount of emulsifier can further be in a range of about 17 wt. % to about 42 wt. %, or 18 wt. % to about 40 wt. %, or 20 wt. % to about 36 wt. %, or 20 wt. % to about 34 wt. %, or about 20 wt. % to about 32 wt. %, or about 20 wt. % to about 30 wt. %, or about 22 wt. % to about 28 wt. %, or for example about 18 wt. %, about 20 wt. %, about 22 wt. %, about 24 wt. %, about 26 wt. %, about 28 wt. %, about 30 wt. %, about 32 wt. %, about 34 wt. %, about 36 wt. %, or about 40 wt. %. The formulation can include about 8 wt. % to about 22 wt. % of an absorption enhancer, optionally one described above, e.g. fatty acid esters of glycerol and PEG esters, e.g. lauroyl polyoxylglycerides (44/14) based on the total weight of the fill material in the hard capsule shell. The amount of absorption enhancer can further be in a range of about 8 wt. % to about 20 wt. %, or about 9 wt. % to about 18 wt. %, or about 10 wt. % to about 16 wt. %, for example about 9 wt. %, about 10 wt. %, about 11 wt. %, about 12 wt. %, about 13 wt. %, about 14 wt. %, about 15 wt. %, or about 16 wt. %. The 25-hydroxyvitamin D active can be dissolved in an alcohol carrier, e.g. ethanol, which is present in the formulation in an amount of about 0.2 wt. % to about 6 wt. %, or about 0.5 wt. % to about 5 wt. %, or about 1 wt. % to about 4 wt. %, or about 2 wt. % to about 4 wt. %, for example about 1.5 wt. %, or about 2.0 wt. % or about 2.5 wt. %, or about 3 wt. %, or about 3.5 wt. %, or about 4 wt. %. The formulation can include a small amount of a preservative, e.g. an antioxidant, e.g. BHT, e.g. in a range of about 0.005 wt. % to about 1 wt. %, or about 0.01 wt. % to about 0.05 wt. %, e.g. about 0.02 wt. %

In another aspect, the 25-hydroxyvitamin D compound(s) can be administered in the form of a formulation as described in international (PCT) application publication WO 2020/044314 A1, including such formulations suitable for dosing to pediatric patients. Such a formulation can be an extended-release formulation, and further optionally can have delayed release characteristics (e.g., alone, or as a result of use in a gelatinized cellulose ether hard capsule shell according to the disclosure herein).

Such a formulation can include a vitamin D compound, optionally 25-hydroxyvitamin D or calcifediol, embedded in a polymer network. The polymer can be water-insoluble, and optionally swellable. In embodiments, the formulation can be an extended release formulation, e.g. for oral use.

Such a formulation can include a spheronized pellet formulation comprising a vitamin D compound, optionally 25-hydroxyvitamin D or calcifediol, and a pharmaceutically acceptable excipient. In embodiments, the formulation can be an extended release formulation, e.g. for oral use. In embodiments, the formulation can be a delayed release formulation, or a delayed-sustained release formulation. The spheronized pellets can be disposed in the capsule, which is optionally enteric coated. In the alternative, the pellets can be enteric coated.

Such a formulation can include a vitamin D formulation comprising a vitamin D compound, optionally 25-hydroxyvitamin D or calcifediol, dispersed in a fatty acid glyceride mixture. In embodiments, the formulation can be an extended release formulation, e.g. for oral use.

Such a formulation can include a nano/microparticle formulation comprising a vitamin D compound, optionally 25-hydroxyvitamin D or calcifediol, and a pharmaceutically acceptable excipient. In embodiments, the nano/microparticle formulation can provide extended release of the vitamin D compound, e.g. by using an extended release polymer as an excipient.

Such a formulation can include a lipid microparticle formulation comprising a vitamin D compound, optionally 25-hydroxyvitamin D or calcifediol, and a pharmaceutically acceptable lipid. In embodiments, the formulation can be an extended release formulation, e.g. for oral use.

Such a formulation can include a non-pareil seed formulation comprising a vitamin D compound, optionally 25-hydroxyvitamin D or calcifediol, and a pharmaceutically acceptable excipient. In embodiments, the formulation can be an extended release formulation, e.g. for oral use. In embodiments, the excipient can include an extended release polymer coating.

Such a formulation can include a pharmaceutical composition comprising a vitamin D compound, optionally 25-hydroxyvitamin D or calcifediol, and a pharmaceutically acceptable excipient selected from one or more excipients in the group of an absorption enhancer, a spheronizing aid, a water insoluble polymer, and a binder. In embodiments, the formulation can be an extended release formulation, e.g. for oral use.

Such a formulation can include a spray-congealed lipid vitamin D formulation comprising a vitamin D compound, optionally 25-hydroxyvitamin D or calcifediol, an extended release agent, and a surfactant. In embodiments, the formulation can be an extended release formulation, e.g. for oral use.

The shell compositions of either the hard capsule can be, in an embodiment, compositions which are stable in low pH environments.

In one type of embodiment, the 25-hydroxyvitamin or calcifediol fill formulation is an extruded-spheronized, ethylcellulose (EC)-based formulation. The amount of 25-hydroxyvitamin or calcifediol can be any amount to provide a dosage form strength according to the disclosure herein, for example in a range of about 0.01 wt. % to about 1 wt. %, or about 0.01 wt. % to about 0.5 wt. %, or about 0.01 wt. % to about 0.1 wt. %. The amount of EC can in a range of about 5 wt. % to about 60 wt. %, for example, based on the amount of the fill composition, or about 1 wt. % to about 20 wt. %, or about 1 wt. % to about 10 wt. %, or about 2 wt. % to about 10 wt. %, or about 10 wt. % to about 30 wt. %. Additives such as one or more fatty acid glycerides, e.g. glyceryl behenate, may be used as an extended release agent. Such compounds are used as thickening or gelling agents and are suitable as extended release agents and include, for example, glyceryl behenate (e.g. Compritol 888 ATO). It can be added at weight percentages of in a range of 5 wt. % to 25 wt. %, or 5 wt. % to 40 wt. %, or 10 wt. % to 30 wt. %, based on the weight of the fill formulation, wherein higher concentrations are particularly contemplated when glyceryl behenate is the major or sole extended release agent. An absorption enhancer, such as medium-chain triglycerides (e.g., Miglyol 812N) and polyglycolized glycerides (e.g. Gelucire 48/16); spheronization aids, such as microcrystalline cellulose (e.g. Avicel PH 101); diluents and pore formers, such as lactose monohydrate or HPMC; binding aids, such as low viscosity hydroxypropyl methylcellulose (e.g. Methocel K3 Premium LV); lubricants, such as talc powder or glyceryl behenate; flavoring agents, such as caramel; and purified water as a process diluent (e.g. to dissolve binder), can be included. Spheronization aids can be present in a concentration of about 30 wt. % to about 90 wt. %, or about 30 wt. % to about 50 wt. %. Absorption enhancers can be present in a concentration of about 3 wt. % to about 25 wt. %, or about 10 wt. % to about 20 wt. %. Binding aids such as low viscosity hydroxypropyl methylcellulose (e.g. Methocel K3) can be present in a concentration of about 3 wt. % to about 10 wt. %, or about 3 wt. % to about 8 wt. %. Lubricants such as talc can be present in a weight concentration of about 0.5 wt % to about 2 wt. %, or 1 wt. % to about 2 wt. %. Antioxidants, e.g. butylated hydroxytoluene (BHT) can be present in a range of about 0.01 wt. % to about 0.05 wt. %. Flavorants are optional, and can be present in a range of about 0.01 wt. % to about 2 wt. %. Such a formulation can be an extended-release formulation, and further optionally can have delayed release characteristics (e.g., alone, or as a result of use in a gelatinized cellulose ether hard capsule shell according to the disclosure herein).

An example formulation is described below, wherein all percentages are weight percentages based on the total weight of the fill material.

| | |
|---|---|
| Calcifediol API | 0.03% |
| Medium chain triglycerides (e.g. Miglyol 812 N) | 10% |
| Antioxidant (e.g. butylated hydroxytoluene) | 0.03% |
| Microcrystalline cellulose (e.g. Avicel PH101) | 38.44% |
| Ethylcellulose (e.g. Ethocel Standard 10 Premium) | 20% |
| Glyceryl behenate (e.g. Compritol 888 ATO) | 20% |
| low viscosity hydroxypropyl methylcellulose | 5% |
| (e.g. Methocel ® K3 Premium LV) | |
| polyglycolized glycerides (e.g. Gelucire 48/16) | 5% |
| Talc | 1% |
| Flavor | 0.5 |

In any of the embodiments contemplated herein, the dissolution release profile of the formulation can have the characteristics of any one of the examples provided herein below. For example, the formulation can be characterized by a dissolution release profile providing a release of vitamin D compound of less than 30% at 2 hours, greater than 45% at 6 hours, and greater than 80% at 12 hours, and further optionally less than 60% at 6 hours in pH 6.8 medium.

In another type of embodiment, the formulation can be characterized by an in vitro dissolution profile providing release of vitamin D compound of less than 30% at 100 to 140 minutes, greater than 45% at 5 to 7 hours, and greater than 80% at 11 to 13 hours in pH 6.8 medium. In another type of embodiment, the formulation can be characterized by an in vitro dissolution profile providing release of vitamin D compound of less than 30% at 2 hours, greater than 45% at 6 hours, and greater than 80% at 12 hours. In these types of embodiments, optionally the release of vitamin D compound at 5 to 7 hours is less than 60%, or at 6 hours is less than 60% in pH 6.8 medium.

In another type of embodiment, the formulation can be characterized by an in vitro dissolution profile providing release of vitamin D compound of about 20% to about 40% at 2 hours, at least 35% at 6 hours, and at least 70% at 12 hours in pH 6.8 medium. In another type of embodiment, the formulation can be characterized by an in vitro dissolution profile providing release of vitamin D compound of about 25% to about 35% at 2 hours, at least 40% at 6 hours, and at least 75% at 12 hours. In these types of embodiments, optionally the release of vitamin D compound is 75% or less at 6 hours, or 65% or less at 6 hours, or 60% or less at 6 hours, for example in pH 6.8 medium.

In another type of embodiment, the formulation can be characterized by an in vitro dissolution profile providing release of vitamin D compound in a two-stage acid (pH 1.2, 2 hours) then pH 6.8 buffer medium at 37° C. of ≤30% at 2 hours, ≥50% and ≤75% at 6 hours, and ≥80% at 12 hours.

The hard capsule formulations also can be effective in preventing early release of API in the first two hours after administration. The present invention thus comprises an extended release dosage form of calcifediol which has an in vitro dissolution profile under two phase acidic/neutral conditions, e.g. 2 hours at pH 1.0 to 2.0, or 1.1, or 1.2, or 1.5, then with transfer to a buffered aqueous medium at pH 6.5, or 6.8 wherein no more than about 7%, or about 5%, or about 4%, or about 3%, or about 2%, or about 1% of calcifediol is released during the first, two-hour period. In one aspect, the dissolution method can be 2 hours at pH 1.5, then with transfer to pH 6.5 buffered medium. In another aspect, the dissolution method can be 2 hours at pH 1.2, then with transfer to pH 6.8 buffered medium. In another aspect, the dissolution method can be 2 hours at pH 1.1, then with transfer to pH 6.8 buffered medium. For example, the dissolution method can be according to USP-NF method <711> using Apparatus 1 or 2 and Method B (1000 mL of 0.1N HCl at 37° C. for 2 hours, drain and then add 1000 mL of pH 6.8 phosphate buffer), optionally Apparatus 2. Thereafter, in a pH 6.8 buffered medium, the release of calcifediol can be up to about 40% or 36% at 4 hours (measured from the start of the 2-phase dissolution testing procedure), at least 60 or 62% at 6 hours, and at least 80 or 84% at 8 hours. The dissolution conditions can be standard conditions as further described herein.

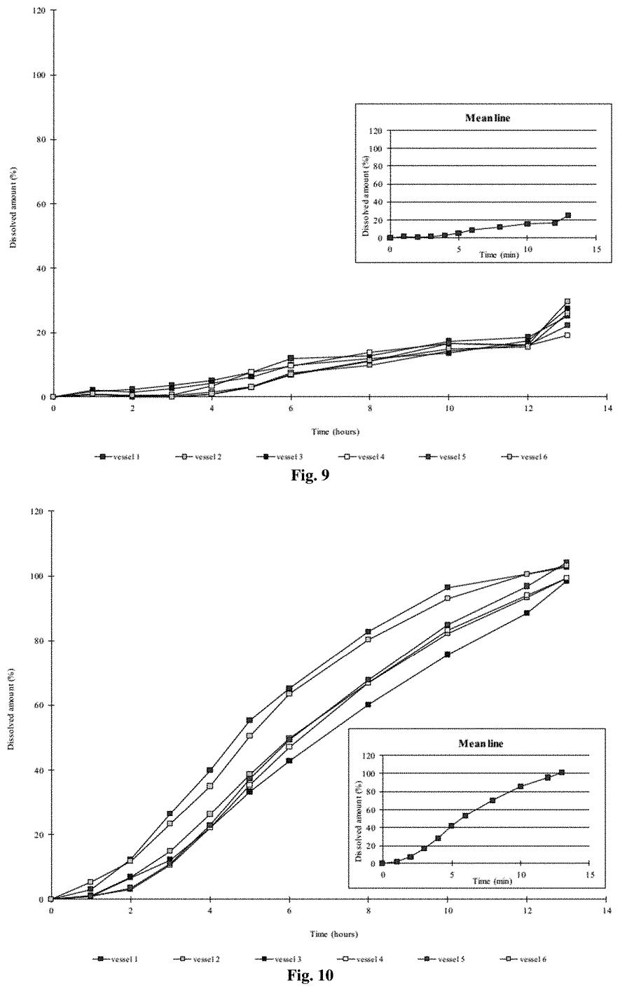

As described in FIG. 2 in connection with Example 2, a gelatinized hypromellose hard shell capsule dosage form according to the disclosure herein was shown to resist dissolution under acid conditions for up to two hours. The release profile in this test shows an approximate 5% release at the 1 hour mark, while the measured amount of release at the 2 hour time point is lower. Without intending to be bound by any particular theory, two possibilities are contemplated. According to the first theory, the higher release at the 1 hour time point is an anomalous variation possibility influenced by early rupture of a single capsule. However, FIG. 9 in connection with Example 6 appears to show a similar behavior for Rayaldee® (calcifediol) extended-release capsules. Accordingly, according to another theory it is possible that both the hard capsule and vegetable (carrageenan)-based capsule shells can exhibit a higher rate of diffusion of active at early time points, before the shell materials (or a component thereof) is fully swollen, while swelling of the capsule shell subsequently slows the rate of diffusion.

In various aspects, the 25-hydroxyvitamin D compound is administered in a modified release formulation. As used herein, the terms “controlled release,” and “modified release” are used interchangeably and refer to the release of the administered vitamin D compound in a way that deviates from immediate release. The modified release formulation can be an extended release formulation. Optionally, the modified release formulation can include a delayed release aspect. As used herein, the terms “sustained release,” “extended release,” and “prolonged release” are used interchangeably and refer to the release of the administered vitamin D compound over a longer period of time than a comparable immediate release formulation.

A hard capsule formulation of 25-hydroxyvitamin D can be used to treat any patient in need of 25-hydroxyvitamin D Patients in need of vitamin D supplementation include healthy subjects and subjects at risk for or having vitamin D insufficiency or deficiency, for example, subjects with stage 1, 2, 3, 4 or 5 CKD; infants, children and adults that do not drink vitamin D fortified milk (e g lactose intolerant subjects, subjects with milk allergy, vegetarians who do not consume milk, and breast fed infants); subjects with rickets; subjects with dark skin (e.g., in the U.S., 42% of African American women between 15 and 49 years of age were vitamin D deficient compared to 4% of white women); the elderly (who have a reduced ability to synthesize vitamin D in skin during exposure to sunlight and also are more likely to stay indoors); institutionalized adults (who are likely to stay indoors, including subjects with Alzheimer's disease or mentally ill); subjects who cover all exposed skin (such as members of certain religions or cultures); subjects who always use sunscreen (e.g., the application of sunscreen with an Sun Protection Factor (SPF) of 8 reduces production of vitamin D by 95%, and higher SPFs may further reduce cutaneous vitamin D production); subjects with fat malabsorption syndromes (including but not limited to cystic fibrosis, cholestatic liver disease, other liver disease, gallbladder disease, pancreatic enzyme deficiency, Crohn's disease, inflammatory bowel disease, sprue or celiac disease, or surgical removal and/or bypass of part or all of the stomach and/or intestines); subjects with inflammatory bowel disease; subjects with Crohn's disease; subjects who have had small bowel resections; subjects with gum disease; subjects taking medications that increase the catabolism of vitamin D, including phenytoin, fosphenytoin, phenobarbital, carbamazepine, and rifampin; subjects taking medications that reduce absorption of vitamin D, including cholestyramine, colestipol, orlistat, mineral oil, and fat substitutes; subjects taking medications that inhibit activation of vitamin D, including ketoconazole; subjects taking medications that decrease calcium absorption, including corticosteroids; subjects with obesity (vitamin D deposited in body fat stores is less bioavailable); subjects with osteoporosis and/or postmenopausal women. According to the Institute of Medicine's report on the Dietary Reference Intakes for vitamin D, food consumption data suggest that median intakes of vitamin D for both younger and older women are below current recommendations; data suggest that more than 50% of younger and older women are not consuming recommended amounts of vitamin D.

In various aspects, the patient's baseline serum total 25-hydroxyvitamin D level can be less than about 30 ng/mL, or less than about 20 ng/mL, or in a range of 20 ng/mL to 30 ng/mL, or in a range of about 20 ng/mL to about 25 ng/mL.

In other aspects, the compositions and methods of the invention are useful for prophylactic or therapeutic treatment of vitamin D-responsive diseases, i.e., diseases where vitamin D, 25-hydroxyvitamin D or active vitamin D (e.g., 1,25-dihydroxyvitamin D) prevents onset or progression of disease, or reduces signs or symptoms of disease. Such vitamin D-responsive diseases include cancer (e.g., breast, lung, skin, melanoma, colon, colorectal, rectal, prostate and bone cancer). 1,25-dihydroxyvitamin D has been observed to induce cell differentiation and/or inhibit cell proliferation in vitro for a number of cells. Vitamin D-responsive diseases also include autoimmune diseases, for example, type I diabetes, multiple sclerosis, rheumatoid arthritis, polymyositis, dermatomyositis, scleroderma, fibrosis, Grave's disease, Hashimoto's disease, acute or chronic transplant rejection, acute or chronic graft versus host disease, inflammatory bowel disease, Crohn's disease, systemic lupus erythematosis, Sjogren's Syndrome, eczema and psoriasis, dermatitis, including atopic dermatitis, contact dermatitis, allergic dermatitis and/or chronic dermatitis. Vitamin D-responsive diseases also include other inflammatory diseases, for example, asthma, chronic obstructive pulmonary disease, polycystic kidney disease, polycystic ovary syndrome, pancreatitis, nephritis, hepatitis, and/or infection. Vitamin D-responsive diseases have also been reported to include hypertension and cardiovascular diseases. Thus, the invention contemplates prophylactic or therapeutic treatment of subjects at risk of or suffering from cardiovascular diseases, for example, subjects with atherosclerosis, arteriosclerosis, coronary artery disease, cerebrovascular disease, peripheral vascular disease, myocardial infarction, myocardial ischemia, cerebral ischemia, stroke, congestive heart failure, cardiomyopathy, obesity or other weight disorders, lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes mellitus, hyperinsulinemia, impaired glucose tolerance, insulin resistance, diabetic complication including neuropathy, nephropathy, retinopathy, diabetic foot ulcer and cataracts), and/or thrombosis.

Diseases which can benefit from a modulation in the levels of vitamin D compounds, include, but are not limited to: (i) in the parathyroid—hypoparathyroidism, pseudohypo-parathyroidism, secondary hyperparathyroidism; (ii) in the pancreas—diabetes; (iii) in the thyroid—medullary carcinoma; (iv) in the skin—psoriasis; wound healing; (v) in the lung—sarcoidosis and tuberculosis; (vi) in the kidney—chronic kidney disease, hypophosphatemic VDRR, vitamin D dependent rickets; (vii) in the bone—anticonvulsant treatment, fibrogenisis imperfecta ossium, osteitis fibrosa cystica, osteomalacia, osteoporosis, osteopenia, osteosclerosis, renal osteodytrophy, rickets; (viii) in the intestine—glucocorticoid antagonism, idiopathic hypercalcemia, malabsorption syndrome, steatorrhea, tropical sprue; and (ix) autoimmune disorders.

In embodiments, the disease that benefits from a modulation in the levels of vitamin D compounds are selected from cancer, dermatological disorders (for example, psoriasis), parathyroid disorders (for example, hyperparathyroidism and secondary hyperparathyroidism), bone disorders (for example, osteoporosis) and autoimmune disorders. In embodiments, the hard capsule 25-hydroxyvitamin D formulation can be used in treatment of SARS-CoV-2 infection. In embodiments, the hard capsule formulation can be used in treatment secondary hyperparathyroidism in patients having Chronic Kidney disease, optionally Stage 3, 4, or 5 CKD, optionally Stage 3 or 4 CKD, optionally Stage 5 CKD, and optionally patients on hemodialysis. A hard capsule formulation of 25-hydroxyvitamin D can be used in lowering serum iPTH levels.

Without limitation, the formulations and dosage forms described herein may be used to treat patients having chronic kidney disease (stages 3, 4 or 5) and secondary hyperparathyroidism as well as treating vitamin D insufficiency and symptoms related to COVID-19. The formulations are particular useful in controlling release of calcifediol over an extended period of time to achieve efficacious reduction of parathyroid hormone in CKD patients and/or to treat patients infected with SARS-CoV-2.

The administration of 25-hydroxyvitamin D, and treatment of COVID-19, as described herein can be performed in the presence of, or in the absence of, additional therapies. As an example, agents for the potentiation of vitamin D action can be administered, e.g. CYP24 inhibitors which can slow the catabolism of 25-hydroxyvitamin D compounds and 1,25-dihydroxyvitamin D compounds.

The subject for therapy or treatment with a formulation as described herein can be a mammal, preferably a human.

With regard to the presently disclosed methods, the amount of the 25-hydroxyvitamin D compound is effective to achieve and maintain a serum total 25-hydroxyvitamin D level of at least 50 ng/mL in the subject during the treatment period. Optionally, the amount is effective to achieve and maintain a serum total 25-hydroxyvitamin D level of at least 60 ng/mL during the treatment period. The method can include achieving such serum levels, e.g. at least or greater than 50 ng/mL, or at least or greater than 60 ng/mL, in the first 24 hours of treatment. The serum level during treatment can be 200 ng/mL or less, or 100 ng/mL or less, in embodiments. For example, the method can include achieving a serum level of at least 50 ng/mL and less than 100 ng/mL in the first 24 hours of treatment. In various instances, the amount is effective to achieve and maintain a serum total 25-hydroxyvitamin D level greater than 60 ng/mL in the subject, e.g., greater than 70 ng/mL, greater than 80 ng/mL, greater than 90 ng/mL, greater than 100 ng/mL, greater than 125 ng/mL, greater than 150 ng/mL, greater than 175 ng/mL, greater than 200 ng/mL, greater than 250 ng/mL, greater than 300 ng/mL, greater than 350 ng/mL, greater than 400 ng/mL, greater than 450 ng/mL, or up to 500 ng/mL during the treatment period, or in a range of about 50 ng/mL to about 100 ng/mL, or about 60 ng/mL to about 100 ng/mL, or greater than 60 ng/mL to about 100 ng/mL during the treatment period.

In various aspects, the 25-hydroxyvitamin D compound is administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly.

In instances, the method of using a hard capsule formulation as described herein includes a loading dose of the 25-hydroxyvitamin D compound administered to the subject before one or more maintenance doses of the 25-hydroxyvitamin D compound. In various aspects, the loading dose is greater than about 90 μg, or at least 100 μg, or at least 200 μg, or at least 250 μg, or greater than about 250 μg or greater than about 500 μg. Optionally, the loading dose is about 1200 μg or less, 1000 μg or less. In various aspects, the loading dose is about 90 μg to about 250 μg, or about 500 μg to about 900 μg, about 500 μg to about 800 μg, about 500 μg to about 700 μg, about 500 μg to about 600 μg, about 600 μg to about 1000 μg, about 700 μg to about 1000 μg, about 800 μg to about 1000 μg, or about 900 μg to about 1000 μg. In various instances, the loading dose is at least or about 900 μg±90 μg of the 25-hydroxyvitamin D compound. Any of the foregoing doses can be administered in the fasted state, e.g. at least 3 hours following a meal, including at bedtime, and without food. Any of the foregoing doses can be administered as an extended-release oral formulation having a bioavailability of about 25%. In embodiments, the loading dose can be the first dose, e.g. the Day 1 dose. In other embodiments, the loading dose is administered in divided doses, e.g. over a period of one or more days, for example 1 to 5 days, or 2 to 5 days. For example, the loading dose can be administered over a period of two or more days, or three days, e.g. a 900 μg loading dose can be administered as 300 μg per day for Days 1, 2, and 3, followed by maintenance doses as described herein, or a 900 μg loaded dose can be administered as 450 μg per day for Days 1 and 2, followed by maintenance doses as described herein. In embodiments, the loading dose is administered in the fasting state.

In various aspects, the one or more daily maintenance doses is at least 25 μg, or at least 30 μg, or greater than 30 μg, or greater than about 50 μg of the 25-hydroxyvitamin D compound. Optionally, each maintenance dose is less than or about 100 μg of the 25-hydroxyvitamin D compound. In various instances, each maintenance dose is about 50 μg to about 100 μg, about 50 μg to about 80 μg, about 50 μg to about 70 μg, about 50 μg to about 60 μg, about 60 μg to about 100 μg, about 70 μg to about 100 μg, about 80 μg to about 100 μg, or about 90 μg to about 100 μg. In various instances, each maintenance dose is about 60 μg±6 μg of the 25-hydroxyvitamin D compound. Any of the foregoing doses can be administered in the fasted state, e.g. at least 3 hours following a meal, including at bedtime, and without food. Any of the foregoing doses can be administered as an extended-release oral formulation having a bioavailability of about 25%. In embodiments, the maintenance doses are administered in the fasting state. Maintenance doses can be administered daily, or a daily maintenance dose can be administered in divided doses throughout the day, or an equivalent amount of 25-hydroxyvitamin D can be administered on a frequency less than daily, e.g. 60 μg every other day in place of 30 μg daily, or about 210 μg weekly in place of 30 μg daily.

Loading doses and maintenance doses can further be adjusted based on a subject's body weight, i.e. such that patients having relatively high BMI levels receive relatively more 25-hydroxyvitamin D.

Loading doses and maintenance doses can further be adjusted based on a subject's serum total 25-hydroxyvitamin D level. For example, a patient who is not vitamin D insufficient or deficient but still has a serum total 25-hydroxyvitamin D level below 50 ng/ml or 60 ng/ml can receive a relatively lower amount of loading dose than a subject who is vitamin D insufficient or deficient.

It is contemplated that doses, e.g. loading doses and/or maintenance doses, can be provided in an amount to maintain a subject's serum total 25-hydroxyvitamin D level of at least 40 ng/ml, or at least 50 ng/ml, or at least 60 ng/ml, for example, in a range of 40 ng/ml to 100 ng/ml, or 50 ng/ml to 200 ng/ml, 50 ng/ml to 100 ng/ml, or 60 ng/ml to 100 ng/ml, or 40 ng/ml to 80 ng/ml.

In various instances, the method comprises administering a daily maintenance dose to the subject, optionally, for at least 3 days, 5, days, 1 week, 10 days, 12 days, 13 days, 2 weeks, 19 days, 20 days, 3 weeks, 26 days, 4 weeks, or more. Optionally, the method comprises administering to the subject a loading dose of 900 μg of the 25-hydroxyvitamin D compound followed by daily maintenance doses for at least 1 week, or at least 2 weeks, or at least 19 days, at least 20 days, or at least 26 days. In various instances, each daily maintenance dose can be 60 μg of the 25-hydroxyvitamin D compound. Optionally, such method comprises administering daily maintenance doses for at least 13 days, or at least 2 weeks, or at least 19 days, or at least 20 days, optionally at least 3 weeks, or at least 26 days, or at least 4 weeks, or more.

In the fasting state, a loading dose of 900 μg of Rayaldee® (calcifediol) extended-release capsules (approximately 25% bioavailability) will raise serum total 25-hydroxyvitamin D level within about 10 hours by about 20 ng/mL to 30 ng/mL, depending on the subject's body weight (the higher the body weight, the lower the expected increase in serum total 25-hydroxyvitamin D). Each daily 60 μg maintenance dose of a Rayaldee® (calcifediol) extended-release capsules will increase serum total 25-hydroxyvitamin D by another 0.6 ng/mL. It follows that subjects having a baseline serum total 25-hydroxyvitamin D level of about 25 ng/mL will reach about 45 ng/mL to 55 ng/mL level after the loading dose, about 53-63 ng/mL after 14 days of maintenance dosing, and 61-71 ng/mL after 26 days of maintenance dosing, when administered in the fasting state. In other embodiments, the method and formulation can be selected to provide a serum total 25-hydroxyvitamin D level of at least 50 ng/mL, or at least 60 ng/mL, and up to 200 ng/mL, or up to 100 ng/mL, in the first 24 hours after the initial dose.

In the fed state, serum total 25-hydroxyvitamin D level will increase about 3 to 4 times more after dosing with Rayaldee® (calcifediol) extended-release capsules compared to in the fasting state. For this reason, and to improve consistency in absorption from dosing, it is contemplated that all dosing can occur at bedtime (in the fasting state, defined as at least about 3 hours after the subject's last meal, optionally at least about 4 hours after the last meal).

Doses for other formulations, both oral and via other dosage routes, can be scaled by the person of ordinary skill based on their bioavailability and/or pharmacokinetics. For example, as Rayaldee® (calcifediol) extended-release capsules have approximately 25% bioavailability, a loading dose for another type of formulation having three times the bioavailability can be greater than about 63 μg bioavailable amount of 25-hydroxyvitamin D delivered by the formulation, or greater than about 125 μg bioavailable amount. Optionally, the loading dose is less than about 250 μg bioavailable amount of 25-hydroxyvitamin D delivered by the formulation. In various aspects, the loading dose is about 125 μg to about 300 μg, about 125 μg to about 225 μg, about 125 μg to about 200 μg, about 125 μg to about 175 μg, about 125 μg to about 150 μg, about 150 μg to about 250 μg, about 175 μg to about 250 μg, about 200 μg to about 250 μg, or about 225 μg to about 250 μg bioavailable amount. Similarly the one or more maintenance doses can be at least about 7 μg, or greater than 7 μg, or greater than about 12 μg of bioavailable 25-hydroxyvitamin D Optionally, each maintenance dose is less than or about 25 μg of bioavailable 25-hydroxyvitamin D In various instances, each maintenance dose can be about 12 μg to about 25 μg, about 12 μg to about 20 μg, about 12 μg to about 17 μg, about 12 μg to about 15 μg, about 15 μg to about 25 μg, about 17 μg to about 25 μg, about 20 μg to about 25 μg, or about 22 μg to about 25 μg of bioavailable 25-hydroxyvitamin D. In various instances, each maintenance dose is about 15 μg±1.5 μg of bioavailable 25-hydroxyvitamin D in such a formulation.

From another perspective, since Rayaldee® (calcifediol) extended-release capsules have a bioavailability of about 25%, dosing amounts can be expressed based on the bioavailable amount of 25-hydroxyvitamin D in any type of formulation. In various aspects, the loading dose is greater than about 22 μg, or at least 25 μg, or at least 50 μg, or at least 62 μg, or greater than about 62 μg or greater than about 125 μg of bioavailable 25-hydroxyvitamin D. Optionally, the loading dose is less than about 250 μg. In various aspects, the loading dose is about 22 μg to about 62 μg, or about 125 μg to about 225 μg, about 125 μg to about 200 μg, about 125 μg to about 175 μg, about 125 μg to about 150 μg, about 150 μg to about 250 μg, about 175 μg to about 250 μg, about 200 μg to about 250 μg, or about 225 μg to about 250 μg. In various instances, the loading dose is at least or about 225 μg±22 μg of bioavailable 25-hydroxyvitamin D. Any of the foregoing doses can be administered in the fasted state, e.g. at least 3 hours following a meal, including at bedtime, and without food. In various aspects, the one or more daily maintenance doses is at least 6 μg, or at least 7 μg, or greater than 7 μg, or greater than about 12 μg of bioavailable 25-hydroxyvitamin D Optionally, each maintenance dose is less than or about 25 μg of bioavailable 25-hydroxyvitamin D In various instances, each maintenance dose is about 12 μg to about 25 μg, about 12 μg to about 20 μg, about 12 μg to about 18 μg, about 12 μg to about 15 μg, about 15 μg to about 25 μg, about 17 μg to about 25 μg, about 20 μg to about 25 μg, or about 22 μg to about 25 μg of bioavailable 25-hydroxyvitamin D In various instances, each maintenance dose is about 15 μg±1.5 μg of the 25-hydroxyvitamin D compound. Any of the foregoing doses can be administered in the fasted state, e.g. at least 3 hours following a meal, including at bedtime, and without food.

Rapidly rising or excessive intracellular levels of vitamin D hormones stimulate the expression of a cytochrome P450 enzyme known as CYP24A1 in cells which contain the vitamin D receptor. The CYP24A1 enzyme catabolizes 1,25-dihydroxyvitamin D, 25-hydroxyvitamin D and vitamin D with high specificity, thereby restoring normal intracellular vitamin D hormone levels. This is an important feedback mechanism that limits excessive and potentially harmful local exposure to vitamin D hormones. Accordingly, it is contemplated to administer 25-hydroxyvitamin D in the absence of upregulating expression of CYP24A1. On the other hand, to provide a rapid response when desired, e.g. an immune response, based on availability of 25-hydroxyvitamin D, e.g. correction of hypovitaminosis D, it is contemplated to safely raise serum total 25-hydroxyvitamin D levels within the first 24 hours of dosing, e.g. to at least 50 ng/mL, or greater than 50 ng/mL, or at least 60 ng/mL, or greater than 60 ng/mL, and optionally up to 200 ng/mL, or up to 100 ng/mL. Similarly, it is contemplated that a formulation for use in the method herein can provide an in vivo Tmax in a range of 4 to 24 hours, or 4 to 18 hours, or 4 to 16 hours, or 4 to 12 hours, or 4 to 8 hours, for example.

A patient's vitamin D metabolite ratio (VMR, calculated as 100 times the ratio of serum 24,25-dihydroxoxyvitamin D₃ to serum 25-hydroxyvitamin D₃, or the ratio of 24,25-dihydroxoxyvitamin D₃ to serum 25-hydroxyvitamin D₃ following administration of a vitamin D₃-type product, e.g. 25-hydroxyvitamin D₃) can be used as an indicator of induction of CYP24A1. See Strugnell SA, Sprague SM, Ashfaq A et al. “Rationale for Raising Current Clinical Practice Guideline Target for Serum 25-Hydroxyvitamin D in Chronic Kidney Disease” Am. J. Nephrol. 2019; 49(4):284-293. Strugnell et al. showed that in Stage 3 and 4 CKD patients having vitamin D insufficiency and SHPT, and treated with 30 or 60 μg ERC over 26 weeks, mean posttreatment VMR rose only moderately (maximum 4.8), suggesting that there was no substantial induction of CYP24A1. Similarly, as described in Example 7 below, in Stage 3 and 4 CKD patients having vitamin D insufficiency and SHPT, and treated with 60 μg ERC for 8 weeks, mean posttreatment VMR also remained below 5 (maximum about 4.2). VMR following dosing with 25-hydroxyvitamin D is dose-dependent. When a sufficiently high dose of 25-hydroxyvitamin D is administered, particularly with immediate-release 25-hydroxyvitamin D, VMR can achieve higher levels. Likewise, with sufficiently frequent repeated dosing with 25-hydroxyvitamin D, VMR can increase over time, and achieve higher levels than desired. In addition, with a sufficiently rapid and potent delivery of 25-hydroxyvitamin D, the rate of VMR increases proportionally. Accordingly, in one aspect, the methods of treatment herein optionally will employ a dosing regimen in which VMR remains substantially constant over a period of at least 28 days, further optionally during maintenance dosing period. In another aspect, the methods of treatment herein optionally will employ a dosing regimen in which VMR decreases over a period of at least 28 days, further optionally during maintenance dosing period. In another aspect, the methods of treatment herein optionally will employ an extended release dosing regimen in which the rate of change of VMR, e.g. in a period of 28 days, is less than the rate of change of VMR for a bioequivalent amount of 25-hydroxyvitamin D administered by immediate release. In another aspect, the methods of treatment herein optionally will employ a dosing regimen in which VMR does not exceed 12, or does not exceed 11, or does not exceed 5, or does not exceed 4.8. In other aspect, recognizing that the patients can benefit from correcting vitamin D insufficiency and achieving a serum total 25-hydroxyvitamin D level of at least 50 ng/ml as described herein, the methods of treatment herein optionally will employ a dosing regimen in which VMR can exceed 4.8, or 5, or 11, or 12, during a loading dose phase, and does not exceed 11, or does not exceed 5, or does not exceed 4.8 during a maintenance dosing phase. In still another aspect, the methods of treatment herein optionally will employ a dosing regimen in which VMR does not exceed 12 during a loading dose phase (e.g. is in a range of 4 to 12), and does not exceed 11 during a maintenance dosing phase (e.g. is in a range of 3 to 11).

In any of the formulations described herein, the hard shell capsule dosage form, or a use thereof, can be designed to provide a rise in serum total 25-hydroxyvitamin D of at least 7 ng/ml and no greater than 30 ng/ml within the first 24 hours after the administering, or at least 8 ng/ml and no greater than 16 ng/ml, or at least 10 ng/ml and no greater than 14 ng/ml. Optionally, such rises can be achieved with a in nominal dosage amounts of 25-hydroxyvitamin D or calcifediol of at least 30 μg, or at least 300 μg, or at least 350 μg, or at least 400 μg, for example in a range of 30 μg to 1800 μg, or 450 μg to 1800 μg, or 30 μg to 1000 μg, or 30 μg to 300 μg. From an effective dose perspective, accounting for bioavailability, it is contemplated that one type of dosage amount of 25-hydroxyvitamin D or calcifediol can be in a range of about 30 μg to about 130 μg, based on bioavailability in the first 24 hours following dosing. For a formulation having 10% bioavailability calculated over a range of 42 days post-dose, an effective dose can be greater than 45 μg, at least 50 μg, at least 60 μg, at least 70 μg, at least 80 μg, or at least 90 μg, for example in a range of 50 μg to 180 μg or a range of 70 μg to 110 μg. In another type of embodiment, the dosage form provide a rise in serum total 25-hydroxyvitamin D in an adult human of less than 3 ng/ml in the first 24 hours after dosing.

The hard capsule dosage form can be characterized by its bioequivalence to the reference Rayaldee® (calcifediol) extended-release capsules. For example, the hard capsule dosage form can be bioequivalent with Rayaldee® (calcifediol) extended-release capsules according to US FDA standards and in according to the FDA's Draft Guidance on Calcifediol dated March 2021. In summary, for example, the bioequivalence can be assessed in a fasting study using a 900 mcg dose in healthy males and non-pregnant, non-lactating females, using a single-dose, two-treatment, two-period crossover in vivo. In another aspect, the bioequivalence can be assessed in a fasting study using a 900 mcg dose in healthy males and non-pregnant, non-lactating females, using a single-dose, two-treatment, parallel in vivo. Subjects can optionally have baseline calcifediol concentrations lower than 30 ng/mL. Subjects can optionally have baseline calcifediol concentrations lower than 35 ng/mL. In the alternative, the study can be performed in the fed state. Baseline-corrected calcifediol concentrations can be measured at −12, −6, and 0 hours before dosing, with the mean of those concentrations used for the baseline correction. In one type of embodiment, the hard capsule formulation according to the disclosure herein will achieve 90% confidence interval (CI) of 80% to 125% of the baseline-adjusted Cmax of Rayaldee® (calcifediol) extended-release capsules (35.87 ng/mL, or about 36 ng/ml). In an alternative contemplated, the hard capsule formulation according to the disclosure herein will achieve 90% CI of about 74% to about 136% of the baseline-adjusted Cmax of Rayaldee® (calcifediol) extended-release capsules (35.87 ng/mL, or about 36 ng/ml); optionally in this embodiment the geometric mean baseline-adjusted Cmax for the hard capsule will be 80% to 125% of the Cmax of Rayaldee® (calcifediol) extended-release capsules. The hard capsule formulation according to the disclosure herein can achieve 90% CI of 80% to 125% of the baseline-adjusted AUC(0-∞) of Rayaldee® (calcifediol) extended-release capsules (9418 ngh/mL).

In any use or method of use or treatment described herein, e.g. secondary hyperparathyroidism in Stage 3, Stage 4, or Stage 5 CKD, the dosage form can be delivered to result in a baseline-adjusted steady state Cmax of serum 25-hydroxyvitamin D or calcifediol in a range of 25 ng/ml to 98 ng/ml, e.g. using a 30 μg dose of 25-hydroxyvitamin D or calcifediol, or in a range of 12.5 ng/ml to 104.9 ng/ml of 25-hydroxyvitamin D or calcifediol, e.g. using a 60 μg daily dose of 25-hydroxyvitamin D or calcifediol. Responders (who met the primary endpoint of greater than 30% iPTH decreases from baseline) in two pivotal studies leading to the approval of Rayaldee® (calcifediol) extended-release capsules showed, at the end of six months of treatment with daily Rayaldee® (calcifediol) extended-release capsules, ranges of baseline adjusted steady state serum calcifediol concentrations of 25-98 ng/ml (% CV of 42.18) and 12.5-104.9 ng/ml (% CV of 33.18) for responders who were on 30 mcg daily and for those with doses titrated up to 60 mcg daily, respectively. These wide effective therapeutic concentrations of calcifediol suggest the effect of extended release 25-hydroxyvitamin D is not dependent on Cmax maintaining in a tight range. Furthermore, Rayaldee® (calcifediol) extended-release capsules are used daily in a chronic condition, and the Cmax parameter is not seen as critical as that of an immediate release formulation required to treat an acute condition.

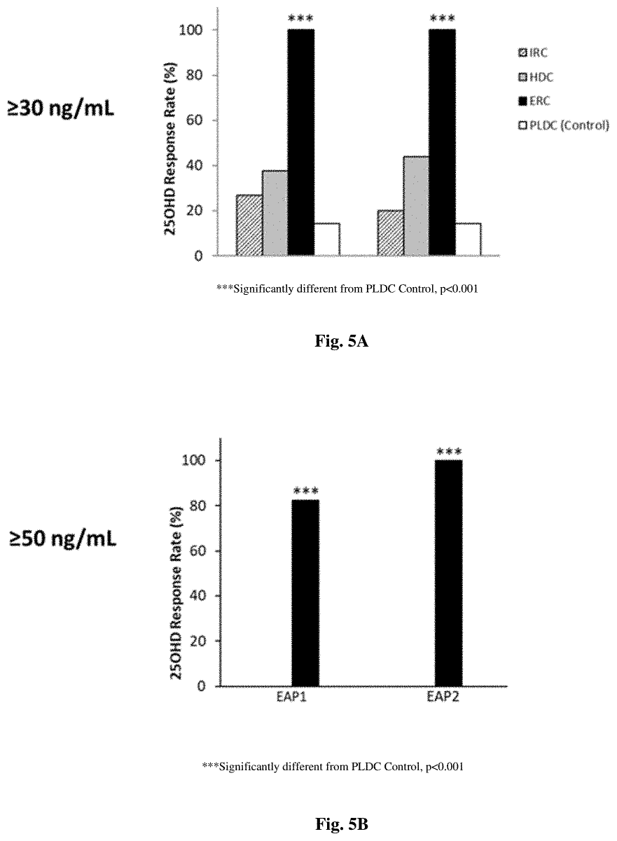

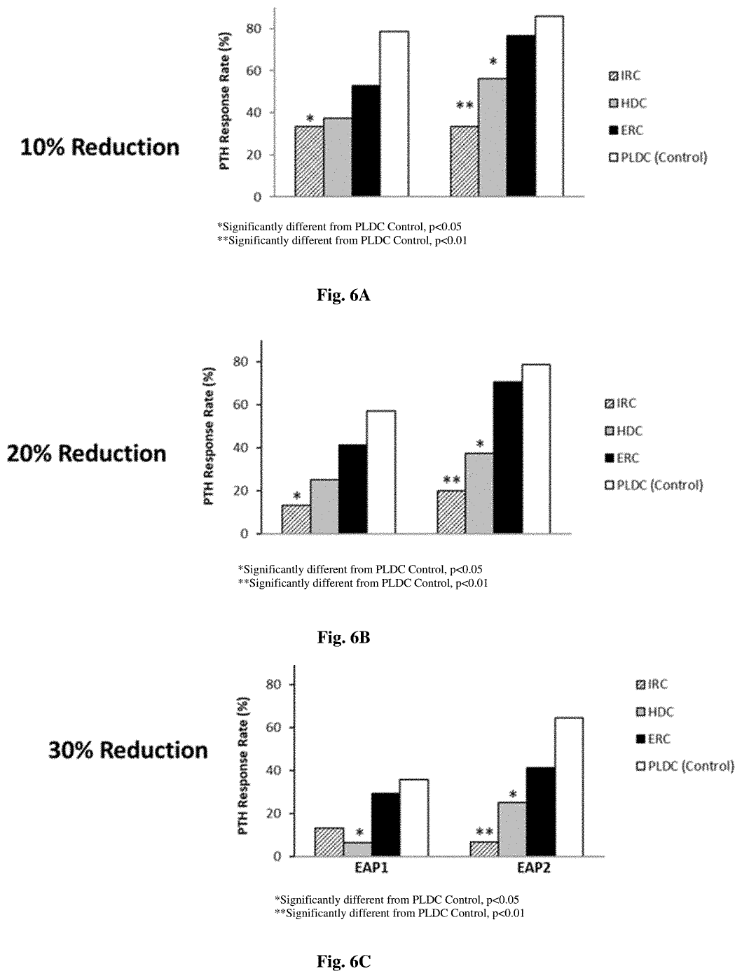

As described in connection with Example 3 below, the Rayaldee® (calcifediol) extended-release capsules (ERC) achieved a serum total 25-hydroxyvitamin D response rate of 100% in both end of the first month of treatment (EAP1) and by the end of the second month of treatment (EAP2) using a target of 30 ng/mL (P<0.001). In contrast, immediate release calcifediol (IRC) had a response rate of 20% in EAP2 using this same target (FIG. 5A). Using a target of 50 ng/mL, ERC achieved response rates above 80% at EAP1 and of 100% at EAP2 (P<0.001) (FIG. 5B), whereas no other treatment was able to raise serum 25-hydroxyvitamin D to this level. Plasma iPTH-lowering responses observed in EAP1 and EAP2 are summarized for all four treatment groups in FIGS. 6A, 6B, and 6C. Response rates for ERC were markedly higher than response rates for IRC. It is expected that a hard cap formulation according to the present disclosure, e.g. one that is made to be bioequivalent to Rayaldee® (calcifediol) extended-release capsules, will show the same benefits over immediate release calcifediol.

As described in connection with Example 4 below, calcifediol was shown to degrade upon exposure to acidic conditions, and especially at higher temperatures, including a temperature of 37° C. characteristic of physiologic conditions. Furthermore, as shown in Example 5 below, regular hypromellose hard shell capsules can dissolve within two hours in such conditions. As described in connection with Example 2 and FIG. 2, a gelatinized hypromellose hard shell capsule dosage form according to the disclosure herein was shown to resist dissolution under such conditions for up to two hours, and more closely match the dissolution release profile of Rayaldee® (calcifediol) extended-release capsules, which include a vegetable based soft capsule shell. Thus, one aspect of the disclosure herein contemplates a use of a gelatinized hard capsule dosage form according to the disclosure herein wherein the dosage form releases no more than about 5% of the 25-hydroxyvitamin D or calcifediol in the formulation contained in the dosage form in two hours in an acidic medium, optionally pH 1.2, or pH 1.5, and further optionally at 37° C., to contain a composition comprising 25-hydroxyvitamin D or calcifediol and provide increased recovery and/or reduced degradation of said 25-hydroxyvitamin D or calcifediol after exposure of the dosage form to acidic conditions. Another aspect contemplates a use of a gelatinized hard capsule dosage form according to the disclosure herein wherein the dosage form releases no more than about 5% of the 25-hydroxyvitamin D or calcifediol in the formulation contained in the dosage form in two hours in an acidic medium, optionally pH 1.2, or pH 1.5, and further optionally at 37° C., to contain a composition comprising 25-hydroxyvitamin D or calcifediol and for oral administration to a mammal Another aspect contemplates a use of a gelatinized hard capsule dosage form according to the disclosure herein wherein the dosage form releases no more than about 5% of the 25-hydroxyvitamin D or calcifediol in the formulation contained in the dosage form in two hours in an acidic medium, optionally pH 1.2, or pH 1.5, and further optionally at 37° C., to contain a composition comprising 25-hydroxyvitamin D or calcifediol and expose the dosage form to acidic conditions, optionally less than pH 4.5, or less than pH 4.0, or less than pH 3.5, or in a range of about pH 1.2 to 3.5, or about pH 1.5 to 3.5, for example.

The methods are contemplated to include embodiments including any combination of one or more of the additional optional elements, features, and steps further described below (including those shown in the figures), unless stated otherwise.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “comprising” indicates the potential inclusion of other agents, elements, steps, or features, in addition to those specified.

### EXAMPLES

The following examples are provided for illustration and are not intended to limit the scope of the invention.

### Example 1

Provided in the table below are HPMC hard capsule formulations of 25-hydroxyvitamin D with varied percentages of paraffin wax and mineral oil (percentages by weight), and the related in vitro dissolution release rates.

| | 0% P. wax | 10% P. wax | 20% P. wax | 30% P. wax | 40% P. wax |
|---|---|---|---|---|---|
| Material | % Cap | % Cap | % Cap | % Cap | % Cap |
| calcifediol | 0.0176% | 0.0176% | 0.0176% | 0.0176% | 0.0176% |
| paraffin | 0.00% | 10.00% | 20.00% | 30.00% | 40.00% |
| mineral oil | 55.34% | 45.34% | 35.34% | 25.34% | 15.34% |
| hypromellose K100 | 10.00% | 10.00% | 10.00% | 10.00% | 10.00% |
| mono-and di-glycerides | 22.55% | 22.55% | 22.55% | 22.55% | 22.55% |
| lauroyl polyoxylglycerides | 9.75% | 9.75% | 9.75% | 9.75% | 9.75% |
| dehydrated ethanol | 2.32% | 2.32% | 2.32% | 2.32% | 2.32% |
| BHT | 0.02% | 0.02% | 0.02% | 0.02% | 0.02% |
| Total | 100.00% | 100.00% | 100.00% | 100.00% | 100.00% |

| | Soft Cap 20% | 0% | 10% | 20% | 30% | 40% |
|---|---|---|---|---|---|---|
| Time (h) | wax | Paraffin | Paraffin | Paraffin | Paraffin | Paraffin |
| 0 | 0.0 | 0.0 | 0.0 | 0.0 | 0.0 | 0.0 |
| 2 | 18.0 | 36.2 | 40.2 | 52.7 | 18.7 | 19.5 |
| 4 | 44.4 | 67.2 | 66.6 | 81.2 | 33.5 | 36.3 |
| 6 | 68.7 | 88.6 | 87.6 | 91.3 | 47.3 | 53.6 |
| 8 | 87.2 | 96.3 | 96.0 | 95.7 | 59.2 | 64.4 |
| 10 | 99.8 | 99.8 | 99.6 | 97.2 | 70.9 | 75.5 |
| 12 | 107.1 | 101.3 | 101.3 | 98.9 | 80.4 | 83.7 |

Reducing the paraffin wax below 20% (to 10% and 0%) with a related increase in mineral oil, compared to the 20 wt. % paraffin formulation, did not provide a significantly faster release profile compared to the comparative soft capsule formulation which contained 20% paraffin wax. On the other hand, increasing the paraffin wax above 20% to 30% and 40%, with a related decrease in mineral oil, did show considerable decrease in the in vitro release rates, especially after 2 hour time point.

### Example 2

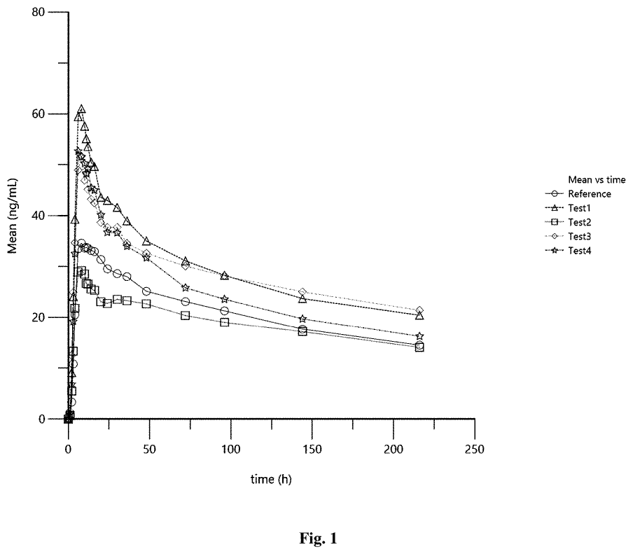

The table below provides examples of additional wax-based hard capsule formulations, a Rayaldee-®-type soft capsule formulation (Reference) with a vegetable-based capsule shell, and modified wax-based soft vegetable-based capsule formulations modified with the goal of providing relatively slower and faster release compared to the Reference formulation. The soft capsules were OptiShell® vegetable-based capsules, containing modified starch and carrageenan. The soft capsule fast (test 1) was formulated to give a fast release rate compared to the Reference by adjusting the concentration of the excipients. The soft capsule fast batch incorporated an increased amount of lauroyl polyoxylglycerides and a reduced amount of paraffin wax. Without intending to be bound by any particular theory, this modification of the matrix properties, to a less solid formulation and a higher concentration of the absorption enhancer compared to the Reference formulation, was intended to enhances the solubility of the active and, as a consequence, increase the release rate as well as the quantity absorbed in vivo, although it did not demonstrate a faster release rate in vitro. The table also includes pharmacokinetic profiles resulting from administering 900 μg doses to each of 16 adult subjects (extracted from mean baseline corrected serum concentration curves, FIG. 1).

| | | | Modified | Modified | | |
|---|---|---|---|---|---|---|
| | | | wax- | wax- | | |
| | | Rayaldee ®- | based | based | HPMC | HPMC |
| | | type soft | soft | soft | Hard | Hard |
| | | capsule | capsule | capsule | capsule | capsule |
| | | (Reference) | (Slow) | (Fast) | (size 3) | (size 4) |
| Excipient | Function | Reference | Test 2 | Test 1 | Test 3 | Test 4 |
| calcifediol | 25- | 0.0176% | 0.0176% | 0.0176% | 0.0176% | 0.0194% |
| | hydroxyvitamin | | | | | |
| | D active | | | | | |
| paraffin wax | control release | 20.00% | 39.00% | 5.00% | 28.00% | 19.95% |
| | agent | | | | | |
| mineral oil | carrier | 35.34% | 30.34% | 45.34% | 27.39% | 35.26% |
| hypromellose | stabilizer | 10.00% | 10.00% | 10.00% | 10.00% | 9.98% |
| mono & | emulsifier | 22.55% | 13.55% | 22.55% | 20.50% | 22.50% |
| diglycerides | | | | | | |
| lauroyl | absorption | 9.75% | 4.75% | 14.75% | 11.75% | 9.73% |
| polyoxyl | enhancer | | | | | |
| glycerides | | | | | | |
| dehydrated | solvent | 2.32% | 2.32% | 2.32% | 2.32% | 2.54% |
| ethanol | | | | | | |
| BHT | antioxidant | 0.02% | 0.02% | 0.02% | 0.02% | 0.02% |
| Total | — | 100.00% | 100.00% | 100.00% | 100.00% | 100.00% |
| Tmax (h) | | 8 | 8 | 8 | 8 | 6 |
| Cmax | | 34.58 | 61.03 | 29.21 | 51.65 | 52.69 |
| (ng/mL) | | | | | | |
| AUC 0-50 h | | 4536.51 | 6347.73 | 4081.29 | 6169.75 | 5385.26 |
| (ng · h · ml) | | | | | | |

FIG. 1 shows related mean serum concentration of 25-hydroxyvitamin D₃ curves after oral administration of 900 μg of the modified release calcifediol soft capsules. The increase of paraffin wax from 20% to 39% did slow the in vitro and in vivo release compared to the Reference, while the decrease of paraffin wax from 20% to 5% did not show fast release rate in vitro under the tested dissolution conditions. This result suggests that below 20% paraffin wax, an erosion mechanism may not be the predominant release mechanism for these formulations. The calcifediol in the fast and the Reference batches was solubilized to the same extent, and adding more emulsifier did not increase the solubility. Increasing the percentage of absorption enhancer from 9.75% to 14.75% had little effect in vitro under the conditions tested, while it increased the absorption in vivo, believed to be through other mechanisms, e.g. tissue penetration. The slow batch in the hard and soft capsules behaved as expected in vitro. The slow batch soft capsules also behaved as expected in vivo. The matrix in this batch is relatively rigid, and the erosion of the active from the rigid matrix is likely the predominant mechanism in this formulation.

The table blow provides dissolution time profiles for the formulations described above, according to USP Apparatus II (paddle with sinker).

| | Time | Time | Dissolution | STD | % |
|---|---|---|---|---|---|
| Formulation | (min) | (h) | (%) | (%) | RSD |
| Reference | 0 | 0 | 0 | 0 | 0 |
| Reference | 60 | 1 | 1.67 | 1.98 | 130.21 |
| Reference | 120 | 2 | 9.04 | 6.24 | 68.99 |
| Reference | 180 | 3 | 20.95 | 9.08 | 43.35 |
| Reference | 240 | 4 | 34.91 | 10.3 | 29.51 |
| Reference | 300 | 5 | 47.96 | 12.47 | 26 |
| Reference | 360 | 6 | 59.45 | 13.02 | 21.9 |
| Reference | 480 | 8 | 76.95 | 9.85 | 12.8 |
| Reference | 600 | 10 | 89.44 | 6.71 | 7.5 |
| Reference | 720 | 12 | 97.1 | 3.87 | 3.98 |
| Test1 | 0 | 0 | 0 | 0 | 0 |
| Test1 | 60 | 1 | 0.275 | 0.52 | 188.652 |
| Test1 | 120 | 2 | 7.564 | 3.089 | 40.833 |
| Test1 | 180 | 3 | 20.331 | 5.441 | 26.764 |
| Test1 | 240 | 4 | 34.255 | 6.303 | 18.4 |
| Test1 | 300 | 5 | 46.652 | 7.61 | 16.312 |
| Test1 | 360 | 6 | 59.015 | 8.374 | 14.19 |
| Test1 | 480 | 8 | 79.576 | 7.948 | 9.988 |
| Test1 | 600 | 10 | 91.199 | 4.816 | 5.281 |
| Test1 | 720 | 12 | 96.886 | 2.22 | 2.291 |
| Test2 | 0 | 0 | 0 | 0 | 0 |
| Test2 | 60 | 1 | 0.933 | 1.518 | 162.768 |
| Test2 | 120 | 2 | 5.823 | 2.687 | 46.141 |
| Test2 | 180 | 3 | 12.406 | 2.572 | 20.728 |
| Test2 | 240 | 4 | 18.377 | 3.092 | 16.826 |
| Test2 | 300 | 5 | 24.532 | 3.394 | 13.835 |
| Test2 | 360 | 6 | 30.788 | 5.149 | 16.723 |
| Test2 | 480 | 8 | 43.173 | 5.999 | 13.895 |
| Test2 | 600 | 10 | 54.453 | 6.125 | 11.248 |
| Test2 | 720 | 12 | 64.16 | 5.505 | 8.58 |
| Test3 | 0 | 0 | 0 | 0 | 0 |
| Test3 | 60 | 1 | 12.028 | 2.806 | 23.331 |
| Test3 | 120 | 2 | 23.336 | 2.622 | 11.234 |
| Test3 | 180 | 3 | 36.166 | 4.912 | 13.581 |
| Test3 | 240 | 4 | 46.122 | 5.707 | 12.373 |
| Test3 | 300 | 5 | 56.73 | 9.602 | 16.927 |
| Test3 | 360 | 6 | 66.205 | 10.866 | 16.412 |
| Test3 | 480 | 8 | 80.356 | 11.287 | 14.046 |
| Test3 | 600 | 10 | 90.945 | 10.168 | 11.18 |
| Test3 | 720 | 12 | 96.979 | 7.576 | 7.812 |
| Test4 | 0 | 0 | 0 | 0 | 0 |
| Test4 | 60 | 1 | 15.107 | 3.614 | 23.92 |
| Test4 | 120 | 2 | 35.029 | 5.557 | 15.863 |
| Test4 | 180 | 3 | 53.623 | 6.598 | 12.304 |
| Test4 | 240 | 4 | 67.86 | 6.892 | 10.156 |
| Test4 | 300 | 5 | 77.733 | 6.345 | 8.162 |
| Test4 | 360 | 6 | 85.366 | 4.774 | 5.592 |
| Test4 | 480 | 8 | 95.639 | 1.746 | 1.826 |
| Test4 | 600 | 10 | 98.212 | 1.582 | 1.611 |
| Test4 | 720 | 12 | 98.653 | 1.876 | 1.902 |

Described in the table below is another hard capsule formulation for 25-hydroxyvitamin D, with a gelatinized HPMC capsule shell. Gellan gum is a hydrophilic polymer and has similar properties to carrageenan used in the vegetable capsule shells of the Reference soft capsule formulation. The gelatinized HPMC capsule has a slower rupture/disintegration time in the stomach than non-gelatinized HPMC capsules.

| Fill Material | % of fill by weight | mg/Cap |
|---|---|---|
| calcifediol | 0.0194% | 0.03 |
| paraffin | 27.95% | 43.32 |
| mineral oil | 32.26% | 50 |
| hypromellose k100 | 9.98% | 15.47 |
| mono-and di-glycerides | 17.5% | 27.13 |
| lauroyl polyoxylglycerides | 9.73% | 15.08 |
| dehydrated ethanol | 2.54% | 3.94 |
| BHT | 0.02% | 0.03 |
| total | 100% | 155 |
| Shell Material | % of shell by weight | mg/Cap |
| hypromellose | qsp100 | 35.283 |
| gellan gum | 5 | 1.9 |
| titanium dioxide | 2 | 10.76 |
| Organic colorant | 0.15 | 0.057 |
| Total | 100 | 38 |

Paraffin wax at a level of 27.95% wax was used instead of 20% as in the Reference soft capsule formulation described above, with slight changes to the mineral oil and mono- and di-glycerides concentrations. The matrix fill was reduced to 155 mg per capsule instead of 170 mg, and the composition was filled in size 4 gelatinized HPMC capsule shells.

The in vivo dissolution profiles (USP Apparatus II (Paddle with Sinker) at 75 RPM, with a medium of 0.5% SDS in 5 mM Sodium Dihydrogenphosphate Monohydrate, pH 6.8, 37±0.5° C., with a volume of 500 ml) for the gelatinized HPMC hard capsule formulation, the Reference soft capsule formulation, and the Test 4 formulation described above are shown in the table below.

| | | | | | Gelatinized HPMC | |
|---|---|---|---|---|---|---|
| | | | | | hard capsule | |
| | Size 4 HC | | Reference | | formulation | |
| | Mean % | | Mean % | | Mean % | |
| Time (hrs) | released | % RSD | released | % RSD | released | % RSD |
| 0 | 0 | N/A | 0.0 | N/A | 0.0 | N/A |
| 1 | 15.1 | 23.9 | | | 11.2 | 19.7 |
| 2 | 35.0 | 15.9 | 10.2 | 35.4 | 25.3 | 17.1 |
| 3 | 53.6 | 12.3 | 37.9 | | 39.2 | 12.7 |
| 4 | 67.9 | 10.2 | 37.9 | 16 | 53.1 | 14.1 |
| 5 | 77.7 | 8.2 | | | 64.3 | 15.4 |
| 6 | 85.4 | 5.7 | 65.8 | 9.7 | 74.3 | 13.5 |
| 8 | 95.6 | 1.8 | 86.1 | 6.4 | 89.0 | 11.8 |
| 10 | 98.2 | 1.6 | 98.5 | 3.2 | 96.3 | 7.7 |
| 12 | 98.7 | 1.9 | 103.7 | 1.3 | 99.4 | 5.2 |

Similarly, a modified in vitro dissolution method was used to measure capsule dissolution (2 capsules in the vessel, 900 ml media, and 60 RPM), the results being shown in the table below (average of 5 Reference batches).

| | Average of 5 batches | | | |
|---|---|---|---|---|
| | Reference capsules | | Gelatinized HMPC hard cap | |
| Time | Mean % | | Mean % | |
| (hrs) | released | % RSD | released | % RSD |
| 0 | 0.0 | N/A | 0.0 | N/A |
| 1 | | | 8.6 | 14.4 |
| 2 | 12.8 | 19.5 | 20.2 | 14.1 |
| 3 | | | | |
| 4 | 41.5 | 11.3 | 43.1 | 12.2 |
| 5 | | | | |
| 6 | 68.2 | 7.5 | 63.5 | 9.8 |
| 8 | 85.6 | 4.3 | 79.7 | 7.6 |
| 10 | 93.4 | 1.8 | 90.0 | 3.8 |
| 12 | 95.7 | 1.4 | 93.0 | 2.9 |
| 14 | | | 93.2 | 2.3 |

A two-stage dissolution method was also used (2 hours at pH 1.2, then transfer to pH 6.8) and the results are shown below and in FIG. 2 (average of 3 reference batches, diamond marker, versus gelatinized HPMC formulation, triangle marker). The dissolution profile of the gelatinized hard capsule formulation closely matches the dissolution profile of the vegetable-based soft capsule formulation.

| Time | Media | Average of 3 Reference soft | gelatinized HPMC hard |
|---|---|---|---|
| (hrs) | pH | capsule batches % released | capsule % released |
| 0 | pH 1.2 | 0.0 | 0 |
| 1 | | 0.3 | 5.0 |
| 2 | | 0.4 | 3.9 |
| 4 | pH 6.8 | 27.9 | 35.7 |
| 6 | | 60.7 | 61.6 |
| 8 | | 78.2 | 84.0 |
| 10 | | 88.7 | 93.0 |
| 12 | | 95.3 | 95.9 |
| 14 | | 98.6 | 96.6 |

The gelatinized HPMC hard capsule formulation and Reference soft capsule formulations are administered to subjects in the fasting state. The pharmacokinetic values and profiles resulting from administration (Cmax, AUC, Tmax) of the gelatinized HPMC hard capsule formulation more closely matches the values and profiles resulting from administration of the Reference formulation, compared to such values and profiles resulting from administration of non-gelatinized HPMC hard capsules, as described above.

### Example 3

A study is carried out with repeated-dosing of ERC (Rayaldee® (calcifediol) extended-release capsules), IR calcifediol (“IRC”), high-dose cholecalciferol (“HDC”), and paricalcitol plus low-dose cholecalciferol (“PLDC”) in adult patients with secondary hyperparathyroidism (SHPT), stage 3 or 4 chronic kidney disease (CKD) and vitamin D insufficiency.

This is an open-label study to gather comparative data evaluating the ERC, IR calcifediol, high-dose cholecalciferol, and paricalcitol plus low-dose cholecalciferol. Eligible subjects were randomized 1:1:1:1 to receive 8 weeks of treatment with one of the listed study medications with a sufficient quantity of a non-alcoholic beverage to enable swallowing of the capsules:
1) ERC capsules 60 μg once daily at bedtime, except on Days 1 and 29 when dosing occurred in the morning before breakfast in the phase 1 unit;
2) IR calcifediol 266 μg before breakfast on the mornings of Day 1 and Day 29 in the phase 1 unit;
3) cholecalciferol 300,000 IU (high-dose) before breakfast on the mornings of Day 1 and Day 29 in the phase 1 unit; and
4) paricalcitol 1 μg (possibly increasing to 2 μg per day at Day 29) plus cholecalciferol 800 IU (low-dose) once daily in the morning before breakfast, except on the mornings of Days 1 and 29 when dosing occurred before breakfast in the phase 1 unit. After 4 weeks of treatment, subjects who received paricalcitol doubled the dose to 2 μg plus cholecalciferol 800 IU once daily in the morning before breakfast provided that (a) the plasma iPTH did not decrease by at least 30% from pretreatment BL and remained above 70 pg/mL, (b) corrected serum calcium is <9.8 mg/dL, and (c) serum phosphorus is <5.5 mg/dL.

The subjects were housed in a phase 1 unit for approximately 14 to 26 hours at the beginning of the study and on study Day 29 to provide the blood samples required.

Subjects receiving treatment with calcitriol or other 1α-hydroxylated vitamin D analog or vitamin D supplementation prior to study completed a 4-week washout period prior to baseline (BL) assessments and remained off these non-study medications for the duration of the study. Subjects were excluded from enrollment if they have received calcimimetic therapy within 12 weeks preceding screening.

Blood samples were collected from all subjects at weekly intervals during the screening and BL periods and during the 8-week treatment period. Subjects maintained a dietary intake during the study of approximately 1,000-1,500 mg of elemental calcium per day by dietary counseling and, if necessary, a prescribed daily calcium supplement.

Subjects reduce the dose of study medication per the schedule below when plasma iPTH is confirmed to be <30 pg/mL, corrected serum calcium is confirmed to be >10.3 mg/dL, or serum phosphorus is confirmed to be >5.5 mg/dL. Subjects suspend dosing if plasma iPTH is confirmed to be <15 pg/mL or corrected serum calcium is confirmed to be >11.0 mg/dL, and resume dosing when plasma iPTH is ≥30 pg/mL and corrected serum calcium is <9.8 mg/dL per the dose schedule below.
ERC: decrease to 30 μg per day (from 60 μg per day)
IR calcifediol: hold Day 29 dose
Cholecalciferol 300,000 IU: hold Day 29 dose
Paricalcitol: decrease dose to 1 μg per day (from 2 μg per day)
Cholecalciferol 800 IU will not be adjusted
   In the event that a dose reduction is required for a subject receiving the minimum dosage of ERC (30 μg per day) or paricalcitol (1 μg per day), the subject will suspend dosing and resume when iPTH is ≥30 pg/mL and corrected serum calcium is <9.8 mg/dL at the same minimum dosage.
Dose resumption (if needed):
ERC: 30 μg per day
Paricalcitol 1 μg per day

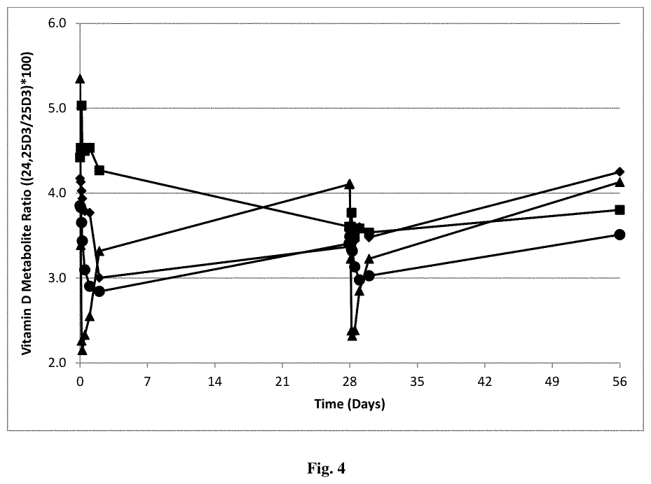

Mean serum total 25-hydroxyvitamin D concentrations as a function of time are shown in FIG. 3 (ERC group with diamonds, IR calcifediol group with triangles, cholecalciferol group with circles, and paricalcitol+cholecalciferol group with squares). The ERC group achieved serum concentrations greater than 50 ng/ml and nearly 90 ng/ml, while the VMR for that group remained below 5 (maximum of mean about 4.2 at end of treatment). VMR as a function of time is shown in FIG. 4.

ERC treatment achieved a serum total 25-hydroxyvitamin D response rate of 100% in both end of the first month of treatment (EAP1) and by the end of the second month of treatment (EAP2) using a target of 30 ng/mL (P<0.001) (FIG. 5A). In contrast, HDC, IRC and PLDC had response rates of 44%, 20% and 14%, respectively, in EAP2 using this same target (FIG. 5A). Using a target of 50 ng/mL, ERC achieved response rates above 80% at EAP1 and of 100% at EAP2 (P<0.001) (FIG. 5B), whereas no other treatment was able to raise serum 25-hydroxyvitamin D to this level. Serum 25-hydroxyvitamin D levels achieved in EAP2 by subjects in all groups showed a significant inverse relationship with body weight and BMI.

Plasma iPTH-lowering responses observed in EAP1 and EAP2 are summarized for all four treatment groups in FIGS. 6A, 6B, and 6C. Response rates for ERC in EAP2 were directionally lower but did not differ significantly from those of PLDC irrespective of whether “response” was defined as a≥10, 20 or 30% reduction from pre-treatment baseline. Response rates with HDC and IRC in EAP2 were even lower (P<0.05). Using the ≥10% threshold for defining “response”, ERC and PLDC had response rates in EAP2 of 76.5% and 85.7%, respectively, which were considerably higher than those in the other two treatment groups (P<0.05) (FIG. 6A). Using the ≥20% threshold, ERC and PLDC had response rates in EAP2 of 70.6% and 78.6%, respectively, compared to response rates of 20.0% and 37.5% for the IRC and HDC groups, respectively (P<0.05) (FIG. 6B). Using the ≥30% threshold, ERC achieved a response rate in EAP2 of 41.2% compared with 64.3% for PLDC (P=NS), 6.7% for IRC (P<0.01) and 25.0% for HDC (P<0.5) (FIG. 6C).

A gelatinized hypromellose capsule dosage form according to the disclosure herein, especially one that is at least substantially bioequivalent to Rayaldee® (calcifediol) extended-release capsules, will exhibit the substantially the same 25-hydroxyvitamin D response rates and plasma iPTH-lowering responses as the ERC dosage form.

### Example 4

The stability of calcifediol was tested in acidic dissolution media at ambient/light conditions and also in dark conditions, at 37° C., 20° C., and 10° C., using 30 μg calcifediol extended release capsules. The results are summarized in the table below. Calcifediol proved to be very unstable in pH 1.2 medium, wherein about 90% degradation was observed at 37° C. after 6 hours. The stability greatly improved after neutralization.

| | | HCl buffer, | Neutralized HCl | Acetate buffer, |
|---|---|---|---|---|
| | | pH = 1.2, | buffer, pH = about | pH = 4.5, |
| Conditions | Time | Recovery % | 4*, Recovery % | Recovery % |
| AS** 10° C. | 20 h | −29.63 | — | 0.34 |
| 20° C. | 20 h | −69.28 | 0.50 | 1.11 |
| | 45 h | — | −1.98 | — |
| | 76 h | — | −6.90 | — |
| 37° C. | 1 h | −31.37 | — | 0.60 |
| | 6 h | −91.13 | — | −0.25 |
| | 13 h | −98.60 | — | −2.12 |
| Ambient | 20 h | −65.86 | — | 1.88 |

*HCl samples were neutralized with sodium acetate and achieved pH about 4
**AS = autosampler. Due to precipitation of SDS, the neutralized samples could not be stored at the autosampler temperature of 10° C.

### Example 5—Comparative Examples

A series of dissolution tests of various calcifediol extended release dosage forms based on different capsule shells was undertaken to better understand release properties in media of different pH conditions, e.g. as in mammalian gastrointestinal tracts, and related effects on the active 25-hydroxyvitamin D ingredient, in this case calcifediol.

5a

The dissolution rate of twelve 30 mcg Rayaldee® (calcifediol) extended-release capsules was monitored in acidic media containing alcohol. Testing was performed in acidic media (0.1 N HCl) at 37° C. using paddles at 100 rpm for 2 hours with pull points every 15 minutes, in 4 groups, each using 12 capsules: (1) no alcohol in the dissolution solution; (2) 5% v/v ethanol in the dissolution solution; (3) 20% v/v ethanol in the dissolution solution; and (4) 40% v/v ethanol. Detection was by UPLC with UV.

No calcifediol was released, even at the 2 hour time point in acidic media and in acidic media containing 5% and 20% ethanol. A small amount of calcifediol (approximate 2-3% of Label Claim 30 mcg) was released in acidic media containing 40% ethanol but at levels below the limit of quantitation (5% of Label Claim for 30 mcg capsules) at all pull points. Visual observations taken during the testing confirmed the results. After 2 hours, it was observed that the blue coating on the original capsules was dissolved, leaving the fill material intact within the sinker at the bottom of the dissolution vessel.

5b

Dissolution testing was carried out on 2 sets of samples, using six replicates in each: (1) 30 mcg Rayaldee® (calcifediol) extended-release capsules; (2) 30 mcg calcifediol capsules based on non-gelatinized hypromellose, containing a Rayaldee® type wax-based fill. The fill was identical in both samples. The dosage form with the hypromellose shell was a size 3 capsule, containing 170 mg fill (same amount of fill as Rayaldee® calcifediol extended release capsules in vegetable-based soft capsule shells).

A dissolution test in pH 6.8 buffered dissolution medium was performed on all samples with sampling points at 1, 2, 3, 4, 5, 6, 7, 8, 10, and 12 hours.

An acid dissolution test was performed on the group (1) samples described above. 500 mL of HCl acid buffer pH 1.2 with 0.5% SDS was used as a dissolution medium. The buffer was prepared according to the USP except that sodium chloride was used instead of potassium chloride to avoid potassium dodecylsulphate precipitation. Due to high instability of calcifediol in acidic solution, the samples were neutralized/stabilized with a sodium acetate solution (150 μL of 273 mg/mL sodium acetate in water) at the time of sample withdrawal, at 1, 2, 3, 4, 5, 6, 7, 8, 10, and 12 hours.

A two stage dissolution test was performed with all samples. The first, acid stage was performed in 500 mL HCl acid buffer pH 1.2 as described in the acid dissolution test immediately above. The sampling time points were at 1 and 2 hours. The samples were also neutralized/stabilized with the same sodium acetate solution described in the acid dissolution test immediately above. After 2 hours, the capsules were removed from the vessels and the medium was replaced with 500 mL of phosphate buffer pH 6.8. The second stage was performed immediately after dissolution medium replacement with the sampling points at 1, 2, 3, 4, 5, 6, 7, 8, 10, and 12 hours thereafter.

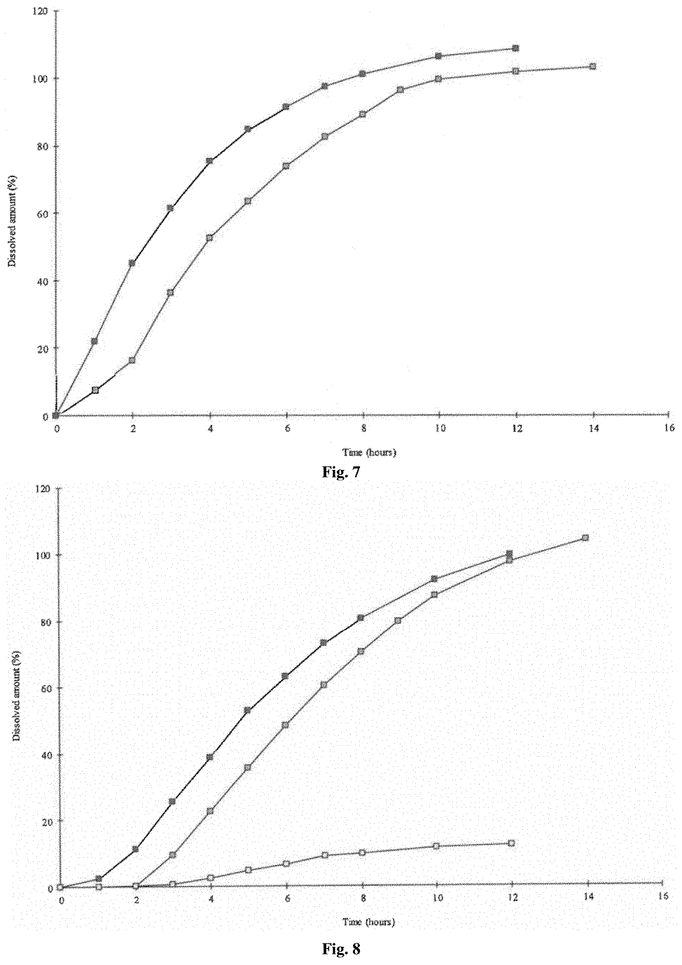

FIG. 7 shows the dissolution profiles for the hypromellose capsule samples (average of six) in pH 6.8 medium (left) and in the two stage dissolution procedure (right). FIG. 8 shows the dissolution profiles for the vegetable capsule samples (average of 6 each) in pH 6.8 medium (left), the two stage dissolution procedure (middle), and pH 1.2 medium (right/bottom).

Shells of the hypromellose capsule dosage forms disintegrated after about 30 to 60 minutes in both dissolution media tested (pH 6.8 and pH 1.2). When the capsule residue was transferred to pH 6.8 medium after the acid stage during the two stage dissolution procedure, the shell seemed to be completely disintegrated, with only capsule fill residue observed in the sinker at that time. No residual fill material remained in any vessels at the end of any of the dissolution testing.

Shells of the 30 mcg Rayaldee® (calcifediol) extended-release capsules disintegrated after about 1 to 2 hours in pH 6.8 dissolution medium and after about 2 hours in acidic medium. During the dissolution medium change in two stage dissolutions, the shells seemed to be completely or almost completely dissolved, with only discontinuous blue coloration (from the original shell color) of the fill surface observed. Small fill residues were detected at the end of pH 6.8 dissolution testing, pH 1.2 testing, and two stage dissolution in some of the vessels.

The dissolution speed of hypromellose capsules in pH 6.8 medium appeared to be faster especially in the first two hours (corresponding to acid stage in the two stage dissolution tests). Final calcifediol release was also higher in pH 6.8 medium than after two stage dissolution. This indicates the possibility that some calcifediol was decomposed during the acid stage. It was not clear from these tests whether the slower dissolution in the acid stage compared to the corresponding period of pH 6.8 medium dissolution can be attributed to the calcifediol decomposition or, for example, to slower disintegration of the shell or slower release from the fill material.

### Example 6—Comparative Example

Rayaldee® (calcifediol) extended-release capsules, 30 μg strength, were tested for dissolution release characteristics in dissolution media of different acidic characteristics.

The dissolution test was run in two different media—hydrochloric acid buffer pH 1.2 and acetate buffer pH 4.5 with addition of 0.5 SDS. The buffer solutions were prepared according to USP (Buffer solutions p. 2165) with one modification: sodium chloride was used instead of potassium chloride in case of hydrochloric acid buffer because of potassium dodecylsulphate precipitation. Due to instability of calcifediol in pH 1.2 medium, these samples were neutralized with the solution of sodium acetate (150 μL of 273 mg/mL solution) at the time of sample withdrawal. The results of the pH 1.2 dissolution test were corrected for the dilution due to the addition of the neutralisation agent.

The test was performed on six replicates in each medium. The dissolution time-points were set as follows: 1-2-3-4-5-6-8-10-12 hours at the nominal paddle speed of 75 RPM, then it was changed to 250 RPM and a sample at 13 h was taken.

The dissolution profiles of the pH 1.2 and 4.5 testing are presented in FIG. 9 and FIG. 10, respectively. the low assay of calcifediol in the pH 1.2 medium is due to the fact that calcifediol is degraded in this medium during the dissolution run.

### Example 7

Two stage dissolution testing was performed on 30 μg strength calcifediol gelatinized hard capsule formulations according to the disclosure herein, and on Rayaldee® (calcifediol) extended-release capsules, 30 μg strength, for comparison.

The hard capsule fill had the following formulation:

| Fill Material | % of fill by weight | mg/Cap |
|---|---|---|
| calcifediol | 0.0194% | 0.03 |
| paraffin | 27.95% | 43.32 |
| mineral oil | 32.26% | 50 |
| hypromellose k100 | 9.98% | 15.47 |
| mono-and di-glycerides | 17.5% | 27.13 |
| lauroyl polyoxylglycerides | 9.73% | 15.08 |
| dehydrated ethanol | 2.54% | 3.94 |
| BHT | 0.02% | 0.03 |
| total | 100% | 155 |
| Shell Material | % of shell by weight | mg/Cap |
| hypromellose | qsp100 | 35.283 |
| gellan gum | 5 | 1.9 |
| titanium dioxide | 2 | 0.76 |
| Organic colorant | 0.15 | 0.057 |
| Total | 100 | 38 |

To also test whether the batch of hot fill formulation changed during the encapsulation procedure, potentially resulting in changes in dissolution characteristics, samples from the start, middle, and end of the encapsulation process were tested separately.

The dissolution apparatus was a USP apparatus 2, paddles with JP sinkers, operated at 75 RPM and at 37±0.5° C. The acid stage dissolution medium was 0.1 N HCl in 0.5% SDS, pH 1.2. 500 mL of 0.5% SDS in 5 nM sodium phosphate buffer was used as the pH 6.8 dissolution medium. One capsule was used per vessel, and 12 replicates were tested. After 2 hours in the acid medium, the capsules were removed from the vessels and the medium was replaced with 500 mL of phosphate buffer pH 6.8.

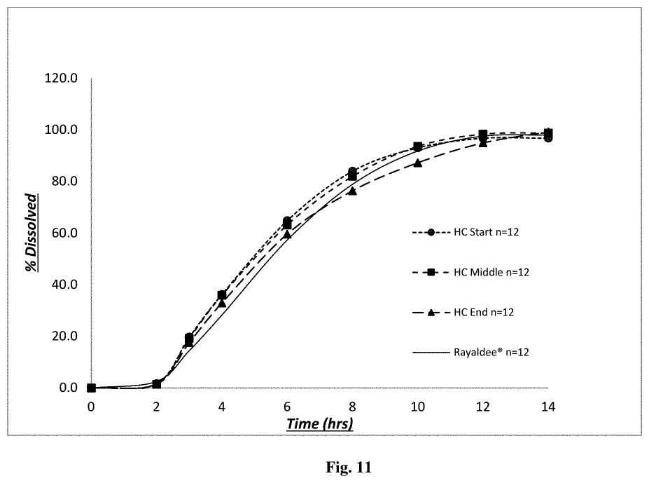

Results are shown in FIG. 11.

### Example 8

A study is conducted to compare oral bioavailability of a calcifediol 30 mcg extended-release hard capsules using gelatinized hypromellose hard shell capsules (Test product) in healthy adult subjects.

The objectives of this study are to compare the bioavailability of the Test product the Reference product (Rayaldee® (calcifediol) extended-release capsules 30 mcg soft capsules) after a single oral dose administration of 900 mcg under fasting conditions; to evaluate and compare the effect of food on the bioavailability of the Test product; and to estimate the Reference-to-Reference intrasubjectCV. The primary study endpoints are the pharmacokinetic (PK) parameters Cmax AUC0-336 of baseline-adjusted calcifediol.

The study is designed as a single center, randomized, single dose, laboratory-blinded, study. Eligible subjects are randomized 2:2:1:1 as follows:

| Sequence (n) | Period 1 | Wash-out | Period 2 |
|---|---|---|---|
| Seq 1 (n = 48) | Treatment-1 | 8 weeks | Treatment-2 |
| | (Test fast) | | (Reference fast) |
| Seq 2 (n = 48) | Treatment-2 | | Treatment-1 |
| | (Reference fast) | | (Test Fast) |
| Seq 3 (n = 24) | Treatment-2 | | Treatment-2 |
| | (Reference fast) | | (Reference fast) |
| Seq 4 (n = 24) | Treatment-3 | | |
| | (Test Fed) | | |

Inclusion criteria include body mass index within 18.5 kg/m² to 30.0 kg/m², inclusively; body weight ≥60 kg; non- or ex-smoker; and serum 25-hydroxyvitamin D level ≥10 and <35 ng/mL or ≥25 and ≤87 nmol/L (depending on the units used by the biomedical laboratory).

Exclusion criteria include: intake of calcifediol in the 60 days prior to study drug administration; intake of an IP in the 28 days prior to study drug administration; unable to avoid consumption of foods and beverages with very high vitamin D content from 10 days prior to study drug administration; unable to avoid excessive consumption (i.e., more than once a day) of foods and beverages with relatively high vitamin D content from 10 days prior to study drug administration; travelled to sunny destinations (e.g., south of the United States, and central America) within 28 days prior to study drug administration or planning to travel to such destination during the study, are employed in outdoor occupations, or are planning participation in prolonged outdoor activities during the study; and sunbathing and using tanning beds within 10 days prior to study drug administration.

A single oral 900 mcg dose of calcifediol (30×30 mcg extended-release capsules) will be administered in the morning. For each subject, all scheduled postdose activities and assessments will be performed relative to the time of study drug administration.

An oral dose of the assigned formulation is administered to subjects within 5 minutes. Subjects are administered the study drug up to 3 capsules at a time, with approximately 240 mL of water at ambient temperature, and an additional amount of water up to 240 mL if required. Time of dosing is set as the time when the first capsule is administered to the subject. The complete dosing procedure is completed within 5 minutes, while dosing procedures done up to 2 minutes outside the allowed time window are no considered as protocol deviations but are documented. The start and end times, as well as the volume of water ingested during study drug administration is recorded. The capsules are swallowed whole and not chewed or broken.

Food intake is controlled during each confinement period and for all subjects. Meals served during confinement are low in vitamin D content to reduce exogenous calcifediol levels. The following items are not included in the meals distributed to the participants: fish and seafood; egg yolks; tofu; soya, almond or rice drinks; cheese; mushrooms; beef liver; any products fortified in vitamin D (including fortified milk, orange juice, margarine, cereals). Meals are identical (same vitamin D content) on baseline and dosing days.

For Treatment-1 and Treatment-2 (Fasting Conditions):

Subjects fast overnight (no food or drink except water), for a minimum of 10 hours prior to dosing.

For Treatment-3 (Fed Conditions—High-Fat, High-Calorie Meal):

Following an overnight fast of at least 10 hours, subjects receive a standardized high fat, high calorie meal 30 minutes before drug administration. An example meal consists of 2 eggs fried in butter, 2 strips of bacon, 2 slices of toast with butter, 4 ounces of hash brown potatoes and 8 ounces of whole milk Substitutions in this test meal may be made provided that the meal delivers a similar amount of calories from protein, carbohydrate, and fat and has comparable meal volume and texture. Subjects must eat the total content of this meal in 30 minutes or less.

Subjects are required to fast for at least 4 hours following dosing, after which a standardized lunch is served. A supper, a light snack, and other meals are served at appropriate times thereafter, but not before 9 hours after dosing.

Twenty-seven (27) blood samples are collected for PK assessments. In order to assess baseline concentrations, blood samples are collected 12, 6 and 0.25 hours prior to drug administration (1×6 mL each). Thereafter, blood samples are collected 1, 2, 4, 6, 7, 8, 9, 10, 11, 12, 14, 16, 20, 24, 30, 36, 42, 48, 72, 96, 144, 216, 288 and 336 hours post drug administration (1×6 mL each). For each period, baseline concentrations are measured approximately 12, 6 and 0.25 hours prior to drug administration. The mean of these 3 predose concentrations is used for baseline correction, while any negative values obtained from baseline correction are designated as zero.

The Reference-to-Reference intra-subject standard deviation (s_{wr}) are determined for each of the primary PK parameters (Cₘₐₓ and AUC₀₋₃₃₆ for baseline-adjusted calcifediol).

Subjects (included in sequence 3) who provide evaluable PK data for the two periods of Reference product are included in the calculation of the s_{wr} to determine if the reference-scaled bioequivalence procedure can be used for the assessment of bioequivalence between the Test and Reference products administered under fasting condition (data from subjects included in sequences 1 and 2).

Average Bioequivalence:

In the event that s_{wr} is below 0.294 (equivalent to ISCV=30%), the two one-sided tests procedure to the bioequivalence assessment will be used for the specific parameters meeting the criteria. A mixed-effects model including sequence, period, and treatment as fixed effects and subject nested within sequence as a random effect will be performed for the ln-transformed parameters. The Test product will be considered to be bioequivalent to the Reference product if the ratio of geometric LSmeans with corresponding 90% confidence interval (CI), calculated from the exponential of the difference between the Test and Reference products for the ln-transformed parameter, is within the 80.00 to 125.00% bioequivalence range.

Reference-Scaled Average Bioequivalence:

In the event that the s_{wr} is equal to or greater than 0.294, the reference-scaled procedure to the bioequivalence assessment will be used for the specific parameters meeting the criteria. The same mixed-effects model as defined above will be performed. The Test product will be considered to be bioequivalent to the Reference product if the ratio of geometric LSmeans with corresponding 90% CI is within the widened acceptance criteria determined as exp(±0.893*s_{wr})*100.

As Cₘₐₓ and AUC₀₋₃₃₆ may have different s_{wr} values, the reference-scaled procedure will only be used for the specific PK parameter that has a s_{wr}≥0.294. The two one-sided tests procedure will be used for PK parameters with s_{wr}<0.294.

Baseline-unadjusted calcifediol results are presented as supportive information.

Food Effect Evaluation

The food effect on the baseline-adjusted calcifediol is determined by comparing the Cₘₐₓ and AUC₀₋₃₃₆ obtained for the Test administered under fed condition and the Test administered under fasted condition.

An absence of food effect on the PK profile will be determined through the following procedure: model with food condition as a fixed effect will be performed for ln-transformed parameters. Absence of food effect could be concluded if the ratio of geometric LSmeans with corresponding 90% CI, calculated from the exponential of the difference between Test administered under fed condition and Test administered under fasting condition for the ln-transformed parameters Cₘₐₓ and AUC₀₋₃₃₆, is within the 80.00 to 125.00% range.

Baseline-unadjusted calcifediol results are presented as supportive information. Tₘₐₓ is analyzed descriptively.

The foregoing description is given for clearness of understanding only, and no unnecessary limitations should be understood therefrom, as modifications within the scope of the invention may be apparent to those having ordinary skill in the art.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Thus, throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure will control.

What is claimed is:

1. A hard capsule dosage form comprising a hard shell capsule containing a solid or semi-solid composition comprising a 25-hydroxyvitamin D compound, the hard shell capsule comprising a cellulose ether and a gelatinizing agent.

2. The hard capsule dosage form of claim 1, wherein the cellulose ether comprises a hydroxyalkyl substituted cellulose ether with 1 to 4 carbon atoms in the alkyl chain.

3. The hard capsule dosage form of claim 1, wherein the cellulose ether comprises one or more selected from the group of methyl cellulose, hydroxyethyl cellulose, hydroxypropyl cellulose, hydroxyethylmethyl cellulose, hydroxyethylethyl cellulose, and hydroxypropylmethyl cellulose.

4. The hard capsule dosage form of claim 1, wherein the viscosity of the cellulose either, as a 2% aqueous solution at 20° C., is in a range of 3 cps to 15 cps.

5. The hard capsule dosage form of claim 1, wherein the cellulose ether is present in the hard shell capsule in an amount in a range of about 90% to 99.98% by weight of the hard shell capsule.

6. The hard capsule dosage form of claim 1, wherein the gelatinizing agent is one or more selected from the group of natural seaweeds, natural seed gums, natural plant exudates, natural fruit extracts, bio-synthetic gums, and bio-synthetic processed starch.

7. The hard capsule dosage form of claim 1, wherein the gelatinizing agent is one or more selected from the group of alginates, agar gum, guar gum, locust bean gum (carob), carrageenan (including kappa-carrageenan and/or iota-carrageenan), tara gum, gum arabic, ghatti gum, Khaya grandifolia gum, tragacanth gum, karaya gum, pectin, arabian (araban), xanthan, gellan, starch, Konjac mannan, galactomannan, funoran, exocellular polysaccharides, xanthan, acetan, gellan, welan, rhamsan, furcelleran, succinoglycan, scieroglycan, schizophyflan, tamarind gum, curdlan, pullulan, and dextran.

8. The hard capsule dosage form of claim 1, wherein the gelatinizing agent is one or more selected from the group of gellan gum, carrageenan, pectin, and pullulan.

9. The hard capsule dosage form of claim 1, wherein the gelatinizing agent is present in an amount in a range of 0.01% to 50% by weight of the hard shell capsule.

10. The hard capsule dosage form of claim 1, wherein the gelatinizing agent is present in an amount in a range of 0.1% to 10% by weight of the hard shell capsule.

11. The hard capsule dosage form of claim 1, wherein the hard shell capsule further comprises a gelling promoter.

12. The hard capsule dosage form of claim 11, wherein the gelling promoter comprises a calcium cation, potassium cation, or sodium cation.

13. The hard capsule dosage form of claim 1, wherein the hard shell capsule is in a range of size 3 to size 5 capsule.

14. The hard capsule dosage form of claim 1, wherein the composition comprising the 25-hydroxyvitamin D comprises:
about 20 wt. % to about 36 wt. % of a wax based on the total weight of the fill material in the hard shell capsule;
about 25 wt. % to about 41 wt. % of an oily vehicle based on the total weight of the composition contained in the hard shell capsule.

15. The hard capsule dosage form of claim 14, wherein the composition comprising the 25-hydroxyvitamin D further comprises about 2 wt. % to about 18 wt. % of a release stabilizing agent based on the total weight of the composition contained in the hard shell capsule.

16. The hard capsule dosage form of claim 14, wherein the composition comprising the 25-hydroxyvitamin D further comprises about 10 wt. % to about 26 wt. % of an emulsifier based on the total weight of the composition contained in the hard shell capsule.

17. The hard capsule dosage form of claim 14, wherein the composition comprising the 25-hydroxyvitamin D further comprises about 3 wt. % to about 17 wt. % of an absorption enhancer based on the total weight of the composition contained in the hard shell capsule.

18. The hard capsule dosage form of claim 1, wherein the composition comprising the 25-hydroxyvitamin D comprises a mixture of the following components, wherein all amounts are specified in weight based on the total weight of the composition contained in the hard shell capsule:
calcifediol: about 0.01% to 0.03%, or 0.0194%;
paraffin: about 25% to 30%, or 27.95%;
mineral oil: about 30% to 35%, or 32.26%;
hypromellose k100: about 7% to 13%, or 9.98%;
mono- and di-glycerides: about 14.5% to 20.5%, or 17.5%;
lauroyl polyoxylglycerides: about 7% to 13%, or 9.73%;
dehydrated ethanol, about 2% to 4%, or 2.54%; and
BHT: about 0.05% to 0.05%, or 0.02%.

19. The hard capsule dosage form of claim 18, wherein the amount of 25-hydroxyvitamin D composition contained in the hard shell capsule is less than 170 mg.

20. The hard capsule dosage form of claim 1, wherein the composition comprising the 25-hydroxyvitamin D comprises an extruded-spheronized mixture of the following components, wherein all amounts are specified in weight based on the total weight of the composition contained in the hard shell capsule:
calcifediol: about 0.01% to 1%, or 0.03%;
medium chain triglycerides: about 5% to about 15%, or 10%;
butylated hydroxytoluene: about 0.01% to about 0.05%, or 0.03%;
microcrystalline cellulose: about 30% to about 50%, or 38.44%;
ethylcellulose: about 10% to about 30%, or 20%;
glyceryl behenate: about 10% to about 30%, or 20%;
low viscosity hydroxypropyl methylcellulose: about 3% to about 8%, or 5%;
polyglycolized glycerides: about 3% to about 8%, or 5%;
talc: about 0.5% to about 2%, or 1%; and
flavor: optional.

21. The hard capsule dosage form of claim 1, wherein the hard shell capsule comprises a mixture of the following components, wherein all amounts are specified in weight based on the total dry weight of hard shell capsule:
gellan gum: about 1% to 10%, or 5%;
titanium dioxide: about 0.01% to 4%, or 2%; and
hypromellose: qsp100.

22. The hard capsule dosage form of claim 1, wherein the dosage form releases no more than about 7% of the 25-hydroxyvitamin D in the formulation in two hours in an acidic medium.

23. The hard capsule dosage form of claim 22, wherein the release in acidic medium is measured in pH 1.2 medium at 37° C. for two hours, followed by measuring dissolution in pH 6.8 buffered medium, and the dosage form releases up to 40% of the 25-hydroxyvitamin D in the formulation at the 4 hour time point.

24. The hard capsule dosage form of claim 23, wherein the dosage form releases at least 60% of the 25-hydroxyvitamin D in the formulation at the 6 hour time point.

25. The hard capsule dosage form of claim 24, wherein the dosage form releases at least 80% of the 25-hydroxyvitamin D in the formulation at the 8 hour time point.

26. The hard capsule dosage form of claim 22, wherein the release in acidic medium is measured in pH 1.2 medium at 37° C. for two hours, followed by measuring dissolution in pH 6.8 buffered medium, and the dosage form releases up to 30% of the 25-hydroxyvitamin D in the formulation at the 2 hour time point, ≥50% and ≤75% of the 25-hydroxyvitamin D in the formulation at the 6 hour time point, and ≥80% of the 25-hydroxyvitamin D in the formulation at the 12 hour time point.

27. The hard capsule dosage form of claim 1, wherein the 25-hydroxyvitamin D comprises or consists of calcifediol.

28. A method of delivering 25-hydroxyvitamin D or calcifediol to a subject in need thereof, comprising administering a hard shell capsule formulation of claim 1 to the subject.

29. A hard capsule dosage form comprising a hard shell capsule containing a solid or semi-solid composition comprising calcifediol, the hard shell capsule comprising at least 80% of a hydroxyalkyl substituted cellulose ether with 1 to 4 carbon atoms in the alkyl chain and having viscosity, as a 2% aqueous solution at 20° C., in a range of 3 cps to 15 cps, and a gelatinizing agent selected from the group of gellan gum, carrageenan, pectin, and pullulan.

30. A method of providing increased recovery and/or reduced degradation of said 25-hydroxyvitamin D or calcifediol in a dosage form comprising 25-hydroxyvitamin D or calcifediol after exposure of the dosage form to acidic conditions, comprising disposing a comprising 25-hydroxyvitamin D or calcifediol in a hard shell capsule comprising a cellulose ether and a gelatinizing agent.
